(19) United States
(12) Patent Application Publication
(10) Pub. No.: US 2005/0123588 A1
(43) Pub. Date: Jun. 9, 2005

(54) Deployable multifunctional hemostatic agent

(76) Inventors: Yong Hua Zhu, Redlands, CA (US); Chang Zheng Yang, Nanjing (CN); Wolff M. Kirsch, Redlands, CA (US); Qun-Dong Shen, Nanjing (CN); Yong Hu, (US); Cindy Dickson, Mentone, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/868,201

(22) Filed: Jun. 14, 2004

Related U.S. Application Data
(60) Provisional application No. 60/479,096, filed on Jun. 16, 2003.
(60) Provisional application No. 60/479,097, filed on Jun. 16, 2003.
(60) Provisional application No. 60/531,362, filed on Dec. 19, 2003.

(51) Int. Cl. A61K 9/70

(52) U.S. Cl. 424/443

(57) ABSTRACT

This invention relates to deployable hemostatic materials comprising chitosan fibers upon which hemostatic microporous polysaccharide microspheres and a medicament or biologically active substance are deposited. The hemostatic materials are suitable for use in controlling active bleeding from artery and vein lacerations, sealing femoral artery punctures, and controlling oozing from tissue.

### RELATED APPLICATIONS

This application claims the benefit of U.S. Provisional Application No. 60/479,096, filed Jun. 16, 2003, U.S. Provisional Application No. 60/479,097 filed Jun. 16, 2003, and U.S. Provisional Application No. 60/531,362, filed Dec. 19, 2003.

### FIELD OF THE INVENTION

This invention relates to deployable hemostatic materials comprising chitosan fibers upon which hemostatic microporous polysaccharide microspheres and a medicament or other biologically active substance are deposited. The hemostatic materials are suitable for use in controlling active bleeding from artery and vein lacerations, sealing femoral artery punctures, and controlling oozing from tissue.

### BACKGROUND OF THE INVENTION

Surgical procedures and traumatic injuries are often characterized by massive blood loss. Conventional approaches for dealing with blood loss, such as manual pressure, cauterization, or sutures can be time consuming and are not always effective in controlling bleeding.

Over the years, a number of topical hemostatic agents have been developed to control bleeding during surgical procedures and from traumatic injury. Some hemostatic agents, such as collagen-based powders, sponges, and cloths, are of a particulate nature. Particulate hemostatic agents provide a lattice for natural thrombus formation, but are unable to enhance this process in coagulopathic patients. Microfibrillar collagen, a particulate hemostatic agent, comes in powder form and stimulates the patient's intrinsic hemostatic cascade. However, this agent has been reported to embolize and induce a localized inflammatory response if used during cardiopulmonary bypass. Pharmacologically-active agents such as thrombin can be used in combination with a particulate carrier, for example, as in a gelfoam sponge or powder soaked in thrombin. Thrombin has been used to control bleeding on diffusely bleeding tissue surfaces, but the lack of a framework onto which the clot can adhere has limited its use. The autologous and allogenic fibrin glues can cause clot formation, but do not adhere well to wet tissue and have little impact on actively bleeding wounds.

### SUMMARY OF THE INVENTION

A hemostatic material that is bioabsorbable, that provides superior hemostasis, and that can be fabricated into a variety of forms suitable for use in controlling bleeding from a variety of wounds is desirable. A hemostatic material that is suitable for use in both surgical applications as well as in field treatment of traumatic injuries is also desirable. For example, in vascular surgery, bleeding is particularly problematic. In cardiac surgery, the multiple vascular anastomoses and cannulation sites, complicated by coagulopathy induced by extracorporeal bypass, can result in bleeding that can only be controlled by topical hemostats. Rapid and effective hemostasis during spinal surgery, where control of osseous, epidural, and/or subdural bleeding or bleeding from the spinal cord is not amenable to sutures or cautery, can minimize the potential for injury to nerve roots and reduce the procedure time. In liver surgery, for example, in live donor liver transplant procedures or in the removal of cancerous tumors, there is a substantial risk of massive bleeding. An effective hemostatic material can significantly enhance patient outcome in such procedures. Even in those situations wherein bleeding is not massive, an effective hemostatic material can be desirable, for example, in dental procedures such as tooth extractions, or for abrasions, burns, and the like. In neurosurgery, oozing wounds are common and are difficult to treat.

Accordingly, in a first embodiment a hemostatic material is provided, the material comprising a hemostatic agent and a therapeutic agent deposited on a hemostatic substrate, wherein the hemostatic substrate comprises chitosan.

In an aspect of the first embodiment, the hemostatic agent comprises microporous polysaccharide microspheres.

In an aspect of the first embodiment, the therapeutic agent comprises an anti-inflammatory agent.

In an aspect of the first embodiment, the therapeutic agent comprises an anti-infective agent.

In an aspect of the first embodiment, the therapeutic agent comprises an anesthetic.

In an aspect of the first embodiment, the therapeutic agent comprises a chemotherapy agent.

In an aspect of the first embodiment, the chitosan comprises a fiber.

In an aspect of the first embodiment, the hemostatic material comprises from about 10 wt. % to about 50 wt. % of a hemostatic agent comprising microporous polysaccharide microspheres.

In an aspect of the first embodiment, the hemostatic material comprises a plurality of chitosan fiber layers.

In a second embodiment, a process for preparing a hemostatic material is provided, the process comprising: a) providing a first chitosan fiber layer; b) applying a solution of a weak acid to the first chitosan fiber layer; c) depositing microporous polysaccharide microspheres on the first chitosan fiber layer; d) depositing a therapeutic agent on the first chitosan fiber layer; and e) placing a second chitosan fiber layer atop the first chitosan fiber layer upon which the microporous polysaccharide microspheres and the therapeutic agent are deposited, whereby a hemostatic material is obtained.

In an aspect of the second embodiment, steps a) through e) are repeated a plurality of times.

In an aspect of the second embodiment, the process further comprises compressing the hemostatic material between a first surface and a second surface, and heating the compressed hemostatic material, whereby a dry hemostatic material is obtained.

In an aspect of the second embodiment, the hemostatic material comprises from about 10 wt. % to about 50 wt. % microporous polysaccharide microspheres.

In a third embodiment, a method of controlling bleeding from a venous laceration, a venous puncture, an arterial laceration, or an arterial puncture is provided, the method comprising applying a hemostatic material to the laceration or the puncture, whereby bleeding is controlled, the hemostatic material comprising a hemostatic agent and a therapeutic agent deposited on a hemostatic substrate, wherein the hemostatic substrate comprises chitosan.

In an aspect of the third embodiment, the hemostatic agent comprises microporous polysaccharide microspheres.

In an aspect of the third embodiment, the therapeutic agent is selected from the group consisting of an anti-inflammatory agent, an anti-infective agent, and an anesthetic.

In an aspect of the third embodiment, the chitosan comprises a fiber.

In an aspect of the third embodiment, the hemostatic material comprises from about 10 wt. % to about 50 wt. % of a hemostatic agent comprising microporous polysaccharide microspheres.

In an aspect of the third embodiment, the hemostatic material comprises a plurality of chitosan fiber layers.

In a fourth embodiment, a method of controlling oozing from a wound is provided, the method comprising applying a hemostatic material to the oozing wound, the hemostatic material comprising a hemostatic agent and a therapeutic agent deposited on a hemostatic substrate, wherein the hemostatic substrate comprises chitosan, whereby oozing is controlled.

In an aspect of the fourth embodiment, the chitosan comprises a nonwoven fabric.

In an aspect of the fourth embodiment, the chitosan comprises a sponge.

In an aspect of the fourth embodiment, the hemostatic material comprises a plurality of chitosan fiber layers.

In an aspect of the fourth embodiment, the therapeutic agent is selected from the group consisting of an anti-inflammatory agent, an anti-infective agent, and an anesthetic.

In an aspect of the fourth embodiment, the therapeutic agent comprises a chemotherapy agent.

In an aspect of the fourth embodiment, the wound comprises a tumor bed.

In an aspect of the fourth embodiment, the wound comprises a liver wound.

In an aspect of the fourth embodiment, the wound comprises a brain wound.

In a fifth embodiment, a process for preparing a hemostatic material is provided, the process comprising: a) providing a first chitosan fiber layer; b) applying a solution of a weak acid to the first chitosan fiber layer; c) depositing microporous polysaccharide microspheres on the first chitosan fiber layer; and d) placing a fifth chitosan fiber layer atop the first chitosan fiber layer upon which the microporous polysaccharide microspheres are deposited, whereby a hemostatic material is obtained.

In an aspect of the fifth embodiment, steps a) through d) are repeated a plurality of times.

In an aspect of the fifth embodiment, the process further comprises heating the hemostatic material, whereby liquid is vaporized from the hemostatic material.

In an aspect of the fifth embodiment, the process further comprises drying the hemostatic material.

In an aspect of the fifth embodiment, the process further comprises compressing the hemostatic material between a first surface and a fifth surface; and heating the compressed hemostatic material, whereby a dry hemostatic material is obtained.

In an aspect of the fifth embodiment, the first surface comprises a polytetrafluoroethylene and the fifth surface comprises a release paper.

In an aspect of the fifth embodiment, the hemostatic material comprises from about 10 wt. % to about 50 wt. % microporous polysaccharide microspheres.

### BRIEF DESCRIPTION OF THE DRAWINGS

FIG. 1 depicts red blood cells compacted by hemostatic microporous polysaccharide microspheres.

FIG. 2 depicts the swelling ability of hemostatic microporous polysaccharide microspheres upon contact with water in an open system.

FIG. 3 depicts sealing a femoral artery puncture with a hemostatic sponge. The expandable, absorbable, biologically-compatible chitosan sponge filled with hemostatic microporous polysaccharide microspheres is placed against the puncture wound via an incision in the skin. The hemostatic sponge expands and holds itself in place against the wall of the artery, sealing the puncture.

FIG. 4 schematically depicts a process for obtaining chitosan from shrimp waste.

FIG. 5 schematically depicts an apparatus for preparing chitosan fibers.

FIG. 6 schematically depicts a layered hemostatic material comprising alternate layers of chitosan fiber and hemostatic powder.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

Hemostasis

Hemostasis is the arrest of bleeding, whether by normal vasoconstriction, by an abnormal obstruction, by coagulation, or by surgical means. Hemostasis by coagulation is dependent upon a complex interaction of plasma coagulation and fibrinolytic proteins, platelets, and the blood vasculature. There are three categories of hemostasis: primary hemostasis; secondary hemostasis; and tertiary hemostasis.

Primary hemostasis is defined as the formation of the primary platelet plug. It involves platelets, the blood vessel wall, and von Willebrand factor. Injury to the blood vessel wall is initially followed by vasoconstriction. Vasoconstriction not only retards extravascular blood loss, but also slows local blood flow, enhancing the adherence of platelets to exposed subendothelial surfaces and the activation of the coagulation process. The formation of the primary platelet plug involves platelet adhesion followed by platelet activation then aggregation to form a platelet plug.

In platelet adhesion, platelets adhere to exposed subendothelium. In areas of high shear rate, such as in the microvasculature, this is mediated by von Willebrand factor (vWf), which binds to glycoprotein Ib-IX in the platelet membrane. In areas of low shear rate, such as in the arteries, fibrinogen mediates the binding of platelets to the subendothelium by attaching to a platelet receptor. The adhesion of platelets to the vessel wall activates them, causing the platelets to change shape, to activate the collagen receptor on their surface, and to release alpha and dense granule constituents. The activated platelets also synthesize and release thromboxane A2 and platelet activating factor, which are potent platelet aggregating agonists and vasoconstrictors.

Platelet aggregation involves the activation, recruitment, and binding of additional platelets, which bind to the adhered platelets. This process is promoted by platelet agonists such as thromboxane 2, PAF, ADP, and serotonin. This activation is enhanced by the generation of thrombin—another platelet agonist—through the coagulation cascade. Platelet aggregation is mediated primarily by fibrinogen, which binds to glycoprotein IIb/IIIa on adjacent platelets. This aggregation leads to the formation of the primary platelet plug, and is stabilized by the formation of fibrin.

In secondary hemostasis, fibrin is formed through the coagulation cascade, which involves circulating coagulation factors, calcium, and platelets. The coagulation cascade involves three pathways: intrinsic; extrinsic; and common.

The extrinsic pathway involves the tissue factor and factor VII complex, which activates factor X. The intrinsic pathway involves high-molecular weight kininogen, prekallikrein, and factors XII, XI, IX and VIII. Factor VIII acts as a cofactor (with calcium and platelet phospholipid) for the factor IX-mediated activation of factor X. The extrinsic and intrinsic pathways converge at the activation of factor X. The common pathway involves the factor X-mediated generation of thrombin from prothrombin (facilitated by factor V, calcium and platelet phospholipid), with the production of fibrin from fibrinogen.

The main pathway for initiation of coagulation is the extrinsic pathway (factor VII and tissue factor), while the intrinsic pathway acts to amplify the coagulation cascade. The coagulation cascade is initiated by the extrinsic pathway with the generation/exposure of tissue factor. Tissue factor is expressed by endothelial cells, subendothelial tissue and monocytes, with expression being upregulated by cytokines. Tissue factor then binds to factor VII and this complex activates factor X. Factor X, in the presence of factor V, calcium, and platelet phospholipid, then activates prothrombin to thrombin. This pathway is rapidly inhibited by a lipoprotein-associated molecule referred to as tissue factor pathway inhibitor. However, the small amount of thrombin generated by this pathway activates factor XI of the intrinsic pathway, which amplifies the coagulation cascade.

The coagulation cascade is amplified by the small amounts of thrombin generated by the extrinsic pathway. This thrombin activates the intrinsic pathway by activation of factors XI and VIII. Activated factor IX, together with activated factor VIII, calcium, and phospholipid, referred to as tenase complex, amplify the activation of factor X, generating large amounts of thrombin. Thrombin, in turn, cleaves fibrinogen to form soluble fibrin monomers, which then spontaneously polymerize to form the soluble fibrin polymer. Thrombin also activates factor XIII, which, together with calcium, serves to cross-link and stabilize the soluble fibrin polymer, forming cross-linked fibrin.

Tertiary hemostasis is defined as the formation of plasmin, which is the main enzyme responsible for fibrinolysis. At the same time as the coagulation cascade is activated, tissue plasminogen activator is released from endothelial cells. Tissue plasminogen activator binds to plasminogen within the clot, converting it into plasmin. Plasmin lyses both fibrinogen and fibrin in the clot, releasing fibrin and fibrinogen degradation products.

The preferred embodiments provide compositions and materials that react with the hemostatic system to treat or prevent bleeding. In particular, the compositions and materials of preferred embodiments result in coagulation of blood.

Effective delivery of hemostatic agents to wounds is particularly desirable in the treatment of injuries characterized by arterial or venous bleeding, as well as in surgical procedures where the control of bleeding can become problematic, e.g., large surface areas, heavy arterial or venous bleeding, oozing wounds, and organ laceration/resectioning. The compositions and materials of preferred embodiments can possess a number of advantages in delivery of hemostatic agents to wounds, including but not limited to ease of application and removal, bioadsorption potential, suturability, antigenicity, and tissue reactivity.

Depending upon the nature of the wound and the treatment method employed, the devices of preferred embodiments can be fabricated in various forms. For example, a puff, fleece, or sponge form can be preferable for controlling the active bleeding from artery or vein, or for controlling internal bleeding during laparoscopic procedures. In neurosurgery, where oozing brain wounds are commonly encountered, a sheet form of the hemostatic material can be preferred. Likewise, in oncological surgery, especially of the liver, it can be preferred to employ a sheet form or sponge form of the hemostatic material, which is placed in or on the tumor bed to control oozing. In dermatological applications, a sheet form can be preferred. In closing punctures in a blood vessel, a puff or fleece form is generally preferred. A suture form, especially a microsuture form, can be preferred in certain applications. Despite differences in delivery and handling characteristics of the various forms, the devices are each effective in deploying hemostatic agents to an affected site and rapidly initiating hemostatic plug formation through platelet adhesion, platelet activation, and blood coagulation.

In preferred embodiments, a hemostatic agent is deposited upon a hemostatic substrate. Particularly preferred embodiments employ bioabsorbable microporous polysaccharide microspheres as the hemostatic agent deposited on a chitosan hemostatic substrate. Any suitable method of depositing the hemostatic agent onto the substrate, adhering the hemostatic agent to a substrate, or incorporating the hemostatic agent into a substrate can be employed.

Hemostatic Agent

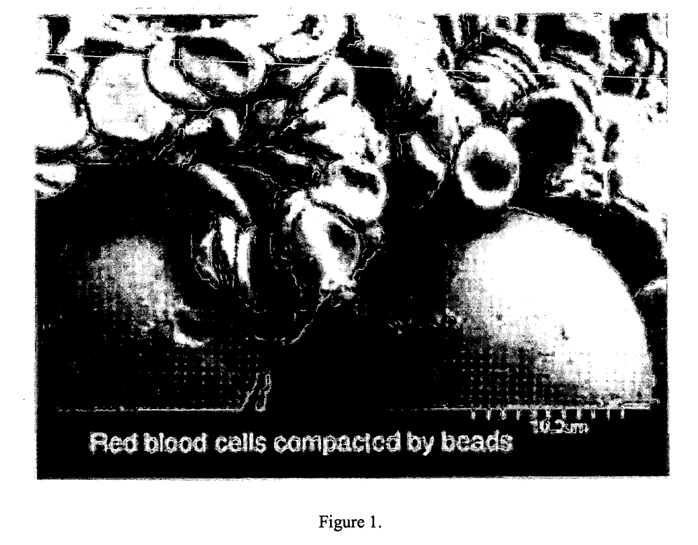

Any suitable hemostatic agent can be deposited upon the substrates of preferred embodiments. However, in a particularly preferred embodiment, the hemostatic agent comprises bioabsorbable microporous polysaccharide microspheres (e.g., TRAUMADEX™ marketed by Emergency Medical Products, Inc. of Waukesha, Wis.). The microspheres have micro-replicated porous channels. The pore size of the microspheres facilitates water absorption and hyperconcentration of albumin, coagulation factors, and other protein and cellular components of the blood. The microspheres also affect platelet function and enhance fibrin formulation. In addition, the microspheres are believed to accelerate the coagulation enzymatic reaction rate. When applied directly, with pressure, to an actively bleeding wound, the particles act as molecular sieves to extract fluids from the blood. The controlled porosity of the particle excludes platelets, red blood cells, and serum proteins larger than 25,000 Daltons, which are then concentrated on the surface of the particles. This molecular exclusion property creates a high concentration of platelets, thrombin, fibrinogen, and other proteins on the particle surface, producing a gelling action. The gelled, compacted cells and constituents accelerate the normal clotting cascade. The fibrin network formed within this dense protein-cell matrix adheres tightly to the surrounding tissue. The gelling process initiates within seconds, and the resulting clot, while exceptionally tenacious, breaks down normally along with the microparticles. FIG. 1 depicts red blood cells compacted by microporous polysaccharide microspheres.

Other suitable hemostatic agents that can be employed in preferred embodiments include, but are not limited to, clotting factor concentrates, recombinant Factor VIIa (NOVOSEVEN®), alphanate FVIII concentrate, bioclate FVIII concentrate, monoclate-P FVIII concentrate, haemate P FVIII, von Willebrand factor concentrate, helixate FVIII concentrate, hemophil-M FVIII concentrate, humate-P FVIII concentrate, hyate-C® Porcine FVIII concentrate, koate HP FVIII concentrate, kogenate FVIII concentrate, recombinate FVIII concentrate, mononine FIX concentrate, and fibrogammin P FXIII concentrate. Such hemostatic agents can be applied to the substrate in any suitable form (powder, liquid, in pure form, in a suitable excipient, on a suitable support or carrier, or the like).

A single hemostatic agent or combination of hemostatic agents can be employed. Preferred loading levels for the hemostatic agent on the substrate can vary, depending upon, for example, the nature of the substrate and hemostatic agent, the form of the substrate, and the nature of the wound to be treated. However, in general it is desirable to maximize the amount of hemostatic agent in relation to the substrate. For example, in the case of a hemostatic puff, a weight ratio of hemostatic agent to substrate of from about 0.001:1 or lower to about 2:1 or higher is generally preferred. More preferably, a weight ratio of hemostatic agent to substrate of from about 0.05:1 or lower to about 2:1 or higher is generally preferred. More preferably, a weight ratio of from about 0.06:1, 0.07:1, 0.08:1, 0.09:1, 0.10:1, 0.15:1, 0.20:1, 0.25:1, 0.30:1, 0.35, 0.40:1, 0.45:1, 0.50:1, 0.55:1, 0.60:1, 0.65:1, 0.70:1, 0.75:1, 0.80:1, 0.85:1, 0.90:1, or 0.95:1 to about 1:1, 1.1:1, 1.2:1, 1.3:1, 1.4:1, or 1.5:1 is employed, although higher or lower ratios can be preferred for certain embodiments.

Hemostatic Substrate

Any suitable hemostatic substrate can be employed as a support for the hemostatic agents of preferred embodiments. However, in a particularly preferred embodiment the hemostatic substrate comprises chitosan. Chitosan is obtained from chitin, a biopolymer obtained principally from shrimp and crab shell waste. Chitosan is the main derivative of chitin, and is the collective term applied to deacetylated chitins in various stages of deacetylation and depolymerization. The chemical structure of chitin and chitosan is similar to that of cellulose. The difference is that instead of the hydroxyl group that is bonded at C-2 in each D-glucose unit of cellulose, there is an acetylated amino group (—NHCOCH₃) at C-2 in each D-glucose unit in chitin and an amino group at C-2 in each D-glucose unit of chitosan.

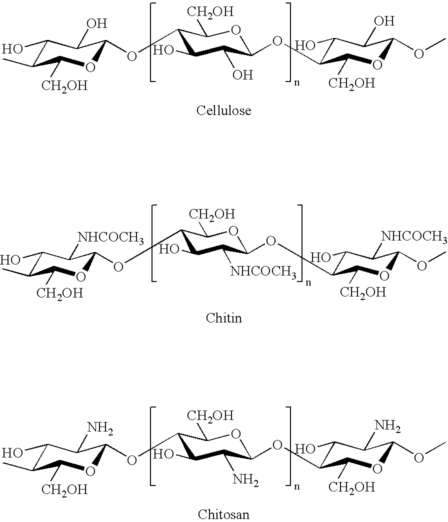

Chitin and chitosan are both nontoxic, but chitosan is used more widely in medical and pharmaceutical applications than chitin because of its superior solubility in acid solution. Chitosan exhibits good biocompatibility and is biodegradable by chitosanase, papain, cellulase, and acid protease. Chitosan exhibits anti-inflammatory and analgesic effects, and promotes hemostasis and wound healing. Chitosan has also been used as a hemostatic agent in surgical treatment and wound protection. The hemostatic effect of chitosan has been described in U.S. Pat. No. 4,394,373.

A single hemostatic substrate or combination of hemostatic substrates of different forms and/or compositions can be employed in the devices of preferred embodiments. Different substrate forms can be preferred, for example, puff, fleece, fabric, sheet, sponge, suture, or powder. A homogeneous mixture of different substrate-forming materials can be employed, or composite substrates can be prepared from two or more different formed substrates. A preferred composite comprises chitosan and collagen.

While chitosan is generally preferred for use as a substrate, other suitable substrates can also be employed. These substrates are preferably bioabsorbable hydrophilic materials that can be fabricated into a desired form (e.g., fiber, sponge, matrix, powder, sheet, suture, fleece, woven fabric, nonwoven fabric, and/or puff).

Other suitable substrates include a synthetic absorbable copolymer of glycolide and lactide. This copolymer is marketed under the trade name VICRYL™ (a Polyglactin 910 manufactured by Ethicon, a division of Johnson & Johnson of Somerset, N.J.). It is absorbed though enzymatic degradation by hydrolysis.

Gelatin sponge is an absorbable, hemostatic sponge used in surgical procedures characterized by venous or oozing bleeding. The sponge adheres to the bleeding site and absorbs approximately forty-five times its own weight in fluids. Due to the uniform porosity of the gelatin sponge, blood platelets are caught within its pores, activating a coagulation cascade. Soluble fibrinogen transforms into a net of insoluble fibrin, which stops the bleeding. When implanted into tissue, the gelatin sponge is absorbed within three to five weeks.

Polyglycolic acid is a synthetic absorbable polymer also suitable for use as a substrate. Polyglycolic acid is absorbed within a few months post-implantation due to its greater hydrolytic susceptibility.

Polylactide is prepared from the cyclic diester of lactic acid (lactide) by ring opening polymerization. Lactic acid exists as two optical isomers or enantiomers. The L-enantiomer occurs in nature, and a D, L racemic mixture results from the synthetic preparation of lactic acid. Fibers spun from polymer derived from the L-enantiomer have high crystallinity when drawn, whereas fibers derived from the racemic mixture are amorphous. Crystalline poly-L-lactide is generally more resistant to hydrolytic degradation than the amorphous D, L form. Hydrolytic degradation rates can be increased by plasticization with triethyl citrate, however the resulting product is less crystalline and more flexible. The time required for poly-L-lactide to be absorbed by the body is relatively long compared to other bioabsorbable materials. Fibers with high tensile strength can be prepared from high molecular weight poly-L-lactide polymers.

Poly(lactide-co-glycolide) polymers are also suitable substrates for use in the preferred embodiments. Copolymers comprising from about 25 to about 70 mole percent glycolide are generally amorphous. Pure polyglycolide is about 50% crystalline, whereas pure poly-L-lactide is about 37% crystalline.

Polydioxanone can be fabricated into fibers to form a substrate suitable for use in preferred embodiments. Polycaprolactone, synthesized from e-caprolactone, is a semi-crystalline polymer absorbed very slowly in vivo. Copolymers of e-caprolactone and L-lactide are elastomeric when prepared from 25% e-caprolactone and 75% L-lactide, and are rigid when prepared from 10% e-caprolactone and 90% L-lactide.

Poly-b-hydroxybutyrate is a biodegradable polymer that occurs in nature and can easily be synthesized in vitro. Poly-b-hydroxybutyrate is also melt processable. Copolymers of hydroxybutyrate and hydroxyvalerate exhibit more rapid degradation than does pure poly-b-hydroxybutyrate.

Synthetic absorbable polyesters containing glycolate ester linkages are suitable for use as substrates in preferred embodiments. Similar copolymers prepared using dioxanone instead of glycolide can also be employed, as can poly(amino acids).

Catgut, siliconized catgut, and chromic catgut are suitable for use as substrates in certain embodiments. However, synthetic materials are generally preferred over natural materials due to their generally predictable performance and reduced inflammatory reaction.

Use of Auxiliary Substances in Preparing Hemostatic Materials

In certain embodiments, it can be desirable to add collagen to the hemostatic agent to accelerate clotting. Other substances that can be added include thrombin, fibrinogen, hydrogels, and oxidized cellulose. Other auxiliary substances can also be employed, as will be appreciated by one skilled in the art.

Multifunctional Hemostatic Materials

In addition to effectively delivering a hemostatic agent to a wound, the hemostatic materials of preferred embodiments can deliver other substances as well. In a particularly preferred embodiment, such substances include medicaments, pharmaceutical compositions, therapeutic agents, and/or other substances producing a physiological effect. The substances can be deposited on the hemostatic substrate by the same methods as are employed to deposit the hemostatic agent, or by any other suitable method as is known in the art for depositing a material on a substrate, or incorporating a material into a substrate.

Medicaments

Any suitable medicament, pharmaceutical composition, therapeutic agent, or other desirable substance can be incorporated into the adhesive formulations of preferred embodiments. Preferred medicaments include, but are not limited to, anti-inflammatory agents, anti-infective agents, anesthetics, and chemotherapy agents.

Suitable anti-inflammatory agents include but are not limited to, nonsteroidal anti-inflammatory drugs (NSAIDs) such as aspirin, celecoxib, choline magnesium trisalicylate, diclofenac potasium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

Anti-infective agents include, but are not limited to, anthelmintics (mebendazole), antibiotics including aminoclycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim.

Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine.

Chemotherapy agents include, but are not limited to, adriamycin, alkeran, Ara-C, BiCNU, busulfan, CCNU, carboplatinum, cisplatinum, cytoxan, daunorubicin, DTIC, 5-FU, fludarabine, hydrea, idarubicin, ifosfamide, methotrexate, mithramycin, mitomycin, mitoxantrone, nitrogen mustard, taxol, velban, vincristine, VP-16, gemcitabine (gemzar), herceptin, irinotecan (camptosar, CPT-11), leustatin, navelbine, rituxan, STI-571, taxotere, topotecan (hycamtin), xeloda (capecitabine), and zevelin.

A variety of other medicaments and pharmaceutical compositions are suitable for use in preferred embodiments. These include cell proliferative agents such as tretinoin, procoagulants such as dencichine (2-amino-3-(oxalylamino)-propionic acid), and sunscreens such as oxybenzone and octocrylene.

Sirolimus (marketed under the tradename Rapamune® by Wyeth-Ayerst, previously referred to as rapamycin) is an immunosuppressive agent suitable for use in preferred embodiments. Sirolimus is a natural macrocyclic lactone with immunosuppressive properties, approved by the FDA in 1999 for the prophylaxis of renal transplant rejection. It has been shown to block T-cell activation and smooth muscle cell proliferation. Sirolimus does not inhibit the endothelialization of the intima. Because of its lipophilicity, the drug penetrates cell membranes enabling intramural distribution and prolonged arterial wall penetration. Cellular uptake is enhanced by binding to the cytosolic receptor, FKBP 12, which also can enhance chronic tissue retention of the drug. Use of sirolimus in cardiac stents for the prevention of restenosis is described in Sousa J E, Costa M A, Abizaid A C, Rensing B J, Abizaid A S, Tanajura L F, Kozuma K, Langenhove G V, Sousa A G M R, Falotico R, Jaeger I, Popma J J, Serruys P W, “Sustained suppression of neointimal proliferation by sirolimus-eluting stents. One-year angiographic and intravascular ultrasound follow-up,” Circulation, 2001, 104:2007-2011; and Marx S O, Marks A R, “Bench to bedside. The development of rapamycin and its application to stent restenosis,” Circulation, 2001, 104:852-855, both of which are incorporated herein by reference in their entirety. Immunosuppresive agents other than sirolimus can also be suitable for use in preferred embodiments.

Human epidermal growth factor (hEGF) can also be preferred for certain embodiments. This small molecular weight peptide is a mitogenic protein and is critical for skin and epidermal regeneration. It is a small 53 amino acid residue long protein with 3 disulfide bridges. This material is available in a salve marketed under the trade name Hebermin™ by Heber Biotech, S.A. of Cuba. The human epidermal growth factor used therein is produced at the Center for Genetic Engineering and Biotechnology, also of Cuba, utilizing recombinant DNA techniques on a generally transformed yeast strain. The epidermal growth factor can be used as produced, or can be polymerized prior to use in preferred embodiments. Presence of hEGF can have a positive effect upon skin healing and regeneration.

Other substances which can be used in preferred embodiments can include, or be derived from, traditional Chinese medicaments, agents, and remedies which have known antiseptic, wound healing, and pain relieving properties. Certain of these agents, though used empirically for many years, are now the subject of intense scientific analysis and research currently being conducted in China at the Nanjing China Pharmaceutical University. These agents include, but are not limited to Sanqi (Radix Notoginsent). One of the compounds in Sanqi is a very effective hemostatic agent called Dencichine. Its chemical composition is as follows:

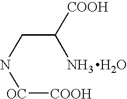

Another such agent is Dahuang (Radix Et Rhizoma Rhei). One of its compounds has anti-inflammatory effect and can also effectively reduce soft tissue edema. The compound is Emodin. Its chemical composition is as follows:

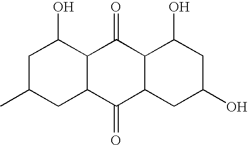

Baiji (Rhizoma Bletillae) has been used as a hemostatic agent and also to promote wound healing for years. It contains the following substances: (3,3′-di-hydroxy-2′, 6′-bis(p-hydroxybenzyl)-5-methoxybibenzyl); 2,6-bis(p-hydroxybenzyl)-3′,5-dimethoxy-3-hydroxy-bibenzyl); (3,3′-dihydroxy-5-methoxy-2,5′,6-tris(p-hydroxy-benzyl) bibenzyl; 7-dihydroxy-1-p-hydroxybenzyl-2-methoxy-9,10-dihydro-phenanthrene); (4,7-dihydroxy-2-methoxy-9, 10-dihydroxyphenanthrene); Blestriarene A (4,4′-dimethoxy-9,9′,10, 10′-tetrahydro[1,1′-biphenanthrene]-2,2′,7,7′-tetrol); Blestriarene B (4,4′-dimethoxy-9, 10-dihydro[1,1′-biphenanthrene]-2,2′,7,7′-tetrol); Batatasin; 3′-O-Methyl Batatasin; Blestrin A(1); Blestrin B(2); Blestrianol A (4,4′-dimethoxy-9,9′,10,10′-tetrahydro]-1′,3-biphenanthrene]-2,2′,7,7′-tetraol); Blestranol B (4′,5-dimethoxy-8-(4-hydroxybenzyl)-9,9′,10,10′-tetrahydro-[1′,3-biphenanthrene]-2,2′,7,7′-tetraol); Blestranol C (4′,5′-dimethoxy-8-(4-hydroxybenzyl)-9,10-dihydro-[1′,3-biphenanthrene]-2,2′,7,7′-tetraol); (1,8-bi(4-hydroxybenzyl)-4-methoxy-phenanthrene-2,7-diol); 3-(4-hydroxybenzyl)-4-methoxy-9,10-dihydro-phenanthrene-2,7-diol; (1,6-bi(4-hydroxybenzyl)-4-methoxy-9,10-dihydro-phenanthrene-2,7-diol; (1-p-hydroxybenzyl-4-methoxyphenanthrene-2,7-diol); 2,4,7-trimethoxy-phenanthrene; 2,4,7-trimethoxy-9,10-dihydrophenanthrene; 2,3,4,7-tetramethoxyphenanthrene; 3,3′,5-trimethoxy-bibenzyl; 3,5-dimethoxybibenzyl; and Physcion.

Rougui (Cortex Cinnamoni) has pain relief effects. It contains the following substances: anhydrocinnzeylanine; anhydrocinnzeylanol; cinncassiol A; cinnacassiol A monoacetate; cinncassiol A glucoside; cinnzeylanine; cinnzeylanol; cinncassiol B glucoside; cinncassiol C₁; cinncassiol C₁ glucoside; cinncassiol C₂; cinncassiol C₂; cinncassiol D₁; cinncassiol D₁ glucoside; cinncassiol D₂; cinncassiol D₂ glucoside; cinncassiol D₃; cinncassiol D₄; cinncassiol D₄ glucoside; cinncassiol E; lyoniresinol; 3α-O—B-D-glucopyranoside; 3,4,5-trimethoxyphenyl 1-O-β-D-apiofuranosyl-(1→6)-β-D-glucopyranoside; (±)-syringaresinol; cinnamic aldehyde cyclic glycerol 1,3 acetals; epicatechin; 3′-O-methy-(−)-epicatechin; 5,3′-di-O-methyl-(−)-epicatechin; 5,7,3′-tri-O-methyl-(−)-epicatechin, 5′-O-methyl-(+)-catechin; 7,4′-di-O-methyl-(+)-catechin; 5,7,4′-tri-O-methyl-(+)-catechin; (−)-epicatechin-3-O-β-D-glucopyranoside; (−)-epicatechin-8-C-β-D-glucopyranoside; (−)-epicatechin-6-C-β-D-glucopyranoside; procyanidin; cinnamtannin A₂, A₃, A₄; (−)-epicatechin; procyanidins B-1, B-2, B-5, B-7, C-1; proanthocyanidin; proanthocyanidin A-2; 8-C-β-D-glucopyranoside; procyanidin B-2 8-C-β-D-glycopyranoside; cassioside [(4s)-2,4-dimethyl-3-(4-hydroxy-3-hydroxymethyl-1-butenyl)-4-(β-D-glucopyranosyl)methyl -2-cyclohexen-1-one]; 3,4,5-trimethoxyphenyl-β-D-apiofuranosyl-1(1→6)-β-D-glucopyranoside; coumarin; cinnamic acid; procyanidin; procyanidin B₂; cinnamoside[(3R)-4-{(2′R,4′ S)-2′-hydroxy-4′-(β-D-apiofuranoxy-(1→6)-β-D-glucopyranosyl)-2′,6′,6′-trimethyl-cyclohexylidene}-3-buten-2-one]; cinnamaldehyde; 3-2(hydroxyphenyl)-propanoic acid; O-glucoside; cinnaman A₂; P, S, Cl, K, Ca, Ti, Mn, Fe, Cu, Zn, Br, Rb, Sr, and Ba.

Zihuaddng (Herba Violae) has been used as an antibiotic agent. Its chemical composition is as follows:

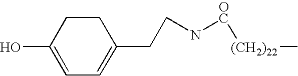

Some of these compounds may be related to epidermal growth factor.

Another compound that can be suitable for use in the preferred embodiments is a carbohydrate with the molecular formula C₁₆H₃₀₂O, which is possibly a quinone, based on the fact that there is one oxygen. This compound has been used for generations for wound healing and pain control. Another compound that is currently being used as a possible hemostatic agent is a substance containing a certain form of seaweed which is commercially available. This seaweed can exert its coagulant effects by the presence of certain collagen and amino acid sequences.

Other substances that can be incorporated into the hemostatic agents of preferred embodiments include various pharmacological agents, excipients, and other substances well known in the art of pharmaceutical formulations. Other pharmacological agents include, but are not limited to, antiplatelet agents, anticoagulants, ACE inhibitors, and cytotoxic agents. These other substances can include ionic and nonionic surfactants (e.g., Pluronic™, Triton™), detergents (e.g., polyoxyl stearate, sodium lauryl sulfate), emulsifiers, demulsifiers, stabilizers, aqueous and oleaginous carriers (e.g., white petrolatum, isopropyl myristate, lanolin, lanolin alcohols, mineral oil, sorbitan monooleate, propylene glycol, cetylstearyl alcohol), emollients, solvents, preservatives (e.g., methylparaben, propylparaben, benzyl alcohol, ethylene diamine tetraacetate salts), thickeners (e.g., pullulin, xanthan, polyvinylpyrrolidone, carboxymethylcellulose), plasticizers (e.g., glycerol, polyethylene glycol), antioxidants (e.g., vitamin E, vitamin C), buffering agents, and the like.

Microencapsulated Medicaments and Auxiliary Substances

In certain embodiments, it can be desirable to provide medicaments, auxiliary substances, or even a portion or all of the hemostatic agent in an encapsulated form to be deposited on the substrate. Certain medicaments, pharmaceutical compositions, therapeutic agents, and other substances desired to be deposited on the substrate can be sensitive to light or air or even the substrate itself, and can be subject to rapid degradation or loss of activity upon exposure to ambient conditions. Other substances may not have sufficient affinity for the substrate to satisfactorily adhere thereto. Microencapsulation is an effective technique to avoid undesired chemical interaction between substances such as medicaments and the substrate or ambient conditions, and can provide superior adhesion to the substrate when compared to the unencapsulated substance.

In a preferred embodiment, antibiotics are entrapped into hydrophilic gelatin or chitosan microcapsules and deposited on the chitosan substrate. Other preferred shell materials include water-soluble alcohols and polyethylene oxides—hydrophilic materials that are expected to exhibit a strong affinity to the hydrophilic chitosan fibers. The microcapsule shell blocks undesired reactions by substantially preventing direct contact of the contents and the substrate, air, or moisture. If an antibiotic is employed in conjunction with the hemostatic agents of preferred embodiments, microencapsulation can permit usage of a spectrum of antibiotics with appropriate sensitivity to different microorganisms. The microencapsulated antibiotics provide long-term controlled release of antibiotics from the hemostatic materials at a preselected concentration.

Microencapsulation techniques involve the coating of small solid particles, liquid droplets, or gas bubbles with a thin film of a material, the material providing a protective shell for the contents of the microcapsule. Microcapsules suitable for use in the preferred embodiments can be of any suitable size, typically from about 1 μm or less to about 1000 μm or more, preferably from about 2 μm to about 50, 60, 70, 80, 90, 100, 200, 300, 400, 500, 600, 700, 800, or 900 μm, and more preferably from about 3, 4, 5, 6, 7, 8, or 9 μm to about 10, 15, 20, 25, 30, 35, 40 or 45 μm. In certain embodiments, it can be preferred to use nanometer-sized microcapsules. Such nanometer-sized microcapsules typically have a size of from about 10 nm or less up to about 1000 nm (1 μm) or more, preferably from about 10, 15, 20, 25, 30, 35, 40, 45, 50, 60, 70, 80, or 90 nm up to about 100, 200, 300, 400, 500, 600, 700, 800, or 900 nm.

While in most embodiments a solid phase medicament or other substance is encapsulated, in certain embodiments it can be preferred to incorporate a liquid or gaseous substance. Liquid or gas containing microcapsules can be prepared using conventional methods well known in the art of microcapsule formation, and such microcapsules can be incorporated into the hemostatic materials of the preferred embodiments.

Microcapsule Components

The microcapsules of preferred embodiments contain a filling material. The filling material is typically one or more medicaments or other pharmaceutical formulations, optionally in combination with substances other than medicaments or pharmaceutical formulations. In certain embodiments, it can be preferred that the microcapsules contain one or more substances not including medicaments or pharmaceutical formulations. The filling material is encapsulated within the microcapsule by a shell material.

Typical shell materials include, but are not limited to, chitin, chitosan, gum arabic, gelatin, ethylcellulose, polyurea, polyamide, aminoplasts, maltodextrins, and hydrogenated vegetable oil. While any suitable shell material can be used in the preferred embodiments, it is generally preferred to use a biodegradable shell material approved for use in food or pharmaceutical applications. Such shell materials include, but are not limited to, gum arabic, gelatin, diethylcellulose, maltodextrins, and hydrogenated vegetable oils. Gelatin is particularly preferred because of its low cost, biocompatibility, and the ease with which gelatin shell microcapsules can be prepared. In certain embodiments, however, other shell materials can be preferred. The optimum shell material can depend upon the particle size and particle size distribution of the filling material, the shape of the filling material particles, compatibility with the filling material, stability of the filling material, and the rate of release of the filling material from the microcapsule.

Microencapsulation Processes

A variety of encapsulation methods can be used to prepare the microcapsules of preferred embodiments. These methods include gas phase or vacuum processes wherein a coating is sprayed or otherwise deposited on the filler material particles so as to form a shell, or wherein a liquid is sprayed into a gas phase and is subsequently solidified to produce microcapsules. Suitable methods also include emulsion and dispersion methods wherein the microcapsules are formed in the liquid phase in a reactor.

Spray Drying

Encapsulation by spray drying involves spraying a concentrated solution of shell material containing filler material particles or a dispersion of immiscible liquid filler material into a heated chamber where rapid desolvation occurs. Any suitable solvent system can be used, however, the method is most preferred for use with aqueous systems. Spray drying is commonly used to prepare microcapsules including shell materials including, for example, gelatin, hydrolyzed gelatin, gum arabic, modified starch, maltodextrins, sucrose, or sorbitol. When an aqueous solution of shell material is used, the filler material typically includes a hydrophobic liquid or water-immiscible oil. Dispersants and/or emulsifiers can be added to the concentrated solution of shell material. Relatively small microcapsules can be prepared by spray drying methods, e.g., from less than about 1 μm to greater than about 50 μm. The resulting particles can include individual particles as well as aggregates of individual particles. The amount of filler material that can be encapsulated using spray drying techniques is typically from less than about 20 wt. % of the microcapsule to more than 60 wt. % of the microcapsule. The process is preferred because of its low cost compared to other methods, and has wide utility in preparing edible microcapsules. The method can not be preferred for preparing heat sensitive materials.

In another variety of spray drying, chilled air rather than desolvation is used to solidify a molten mixture of shell material containing filler material in the form of particles or an immiscible liquid. Various fats, waxes, fatty alcohols, and fatty acids are typically used as shell materials in such an encapsulation method. The method is generally preferred for preparing microcapsules having water-insoluble shells.

Fluidized-Bed Microencapsulation

Encapsulation using fluidized bed technology involves spraying a liquid shell material, generally in solution or melted form, onto solid particles suspended in a stream of gas, typically heated air, and the particles thus encapsulated are subsequently cooled. Shell materials commonly used include, but are not limited to, colloids, solvent-soluble polymers, and sugars. The shell material can be applied to the particles from the top of the reactor, or can be applied as a spray from the bottom of the reactor, e.g., as in the Wurster process. The particles are maintained in the reactor until a desired shell thickness is achieved. Fluidized bed microencapsulation is commonly used for preparing encapsulated water-soluble food ingredients and pharmaceutical compositions. The method is particularly suitable for coating irregularly shaped particles. Fluidized bed encapsulation is typically used to prepare microcapsules larger than about 100 μm, however smaller microcapsules can also be prepared.

Complex Coacervation

A pair of oppositely charged polyelectrolytes capable of forming a liquid complex coacervate (namely, a mass of colloidal particles that are bound together by electrostatic attraction) can be used to form microcapsules by complex coacervation. A preferred polyanion is gelatin, which is capable of forming complexes with a variety of polyanions. Typical polyanions include gum arabic, polyphosphate, polyacrylic acid, and alginate. Complex coacervation is used primarily to encapsulate water-immiscible liquids or water-insoluble solids. The method is generally not suitable for use with water soluble substances, or substances sensitive to acidic conditions.

In the complex coacervation of gelatin with gum arabic, a water insoluble filler material is dispersed in a warm aqueous gelatin emulsion, and then gum arabic and water are added to this emulsion. The pH of the aqueous phase is adjusted to slightly acidic, thereby forming the complex coacervate which adsorbs on the surface of the filler material. The system is cooled, and a cross-linking agent, such as glutaraldehyde, is added. The microcapsules can optionally be treated with urea and formaldehyde at low pH so as to reduce the hydrophilicity of the shell, thereby facilitating drying without excessive aggregate formation. The resulting microcapsules can then be dried to form a powder.

Polymer-Polymer Incompatibility

Microcapsules can be prepared using a solution containing two liquid polymers that are incompatible, but soluble in a common solvent. One of the polymers is preferentially absorbed by the filler material. When the filler material is dispersed in the solution, it is spontaneously coated by a thin film of the polymer that is preferentially absorbed. The microcapsules are obtained by either crosslinking the absorbed polymer or by adding a nonsolvent for the polymer to the solution. The liquids are then removed to obtain the microcapsules in the form of a dry powder.

Polymer-polymer incompatibility encapsulation can be carried out in aqueous or nonaqueous media. It is typically used for preparing microcapsules containing polar solids with limited water solubility. Suitable shell materials include ethylcellulose, polylactide, and lactide-glycolide copolymers. Polymer-polymer incompatibility encapsulation is often preferred for encapsulating oral and parenteral pharmaceutical compositions, especially those containing proteins or polypeptides, because biodegradable microcapsules can be easily prepared. Microcapsules prepared by polymer-polymer incompatibility encapsulation tend to be smaller than microcapsules prepared by other methods, and typically have diameters of 100 μm or less.

Interfacial Polymerization

Microcapsules can be prepared by conducting polymerization reactions at interfaces in a liquid. In one such type of microencapsulation method, a dispersion of two immiscible liquids is prepared. The dispersed phase forms the filler material. Each phase contains a separate reactant, the reactants capable of undergoing a polymerization reaction to form a shell. The reactant in the dispersed phase and the reactant in a continuous phase react at the interface between the dispersed phase and the continuous phase to form a shell. The reactant in the continuous phase is typically conducted to the interface by a diffusion process. Once the reaction is initiated, the shell eventually becomes a barrier to diffusion and thereby limits the rate of the interfacial polymerization reaction. This can affect the morphology and uniformity of thickness of the shell. Dispersants can be added to the continuous phase. The dispersed phase can include an aqueous or a nonaqueous solvent. The continuous phase is selected to be immiscible in the dispersed phase.

Typical polymerization reactants can include acid chlorides or isocyanates, which are capable of undergoing a polymerization reaction with amines or alcohols. The amine or alcohol is solubilized in the aqueous phase in a nonaqueous phase capable solubilizing the amine or alcohol. The acid chloride or isocyanate is then dissolved in the water- (or nonaqueous solvent-) immiscible phase. Similarly, solid particles containing reactants or having reactants coated on the surface can be dispersed in a liquid in which the solid particles are not substantially soluble. The reactants in or on the solid particles then react with reactants in the continuous phase to form a shell.

In another type of microencapsulation by interfacial polymerization, commonly referred to as in situ encapsulation, a filler material in the form of substantially insoluble particles or in the form of a water immiscible liquid is dispersed in an aqueous phase. The aqueous phase contains urea, melamine, water-soluble urea-formaldehyde condensate, or water-soluble urea-melamine condensate. To form a shell encapsulating the filler material, formaldehyde is added to the aqueous phase, which is heated and acidified. A condensation product then deposits on the surface of the dispersed core material as the polymerization reaction progresses. Unlike the interfacial polymerization reaction described above, the method can be suitable for use with sensitive filler materials since reactive agents do not have to be dissolved in the filler material.

In a related in situ polymerization method, a water-immiscible liquid or solid containing a water-immiscible vinyl monomer and vinyl monomer initiator is dispersed in an aqueous phase. Polymerization is initiated by heating and a vinyl shell is produced at the interface with the aqueous phase.

Gas Phase Polymerization

Microcapsules can be prepared by exposing filler material particles to a gas capable of undergoing polymerization on the surface of the particles. In one such method, the gas comprises p-xylene dimers that polymerize on the surface of the particle to form a poly(p-xylene) shell. Specialized coating equipment can be necessary for conducting such coating methods, making the method more expensive than certain liquid phase encapsulation methods. Also, the filler material to be encapsulated is preferably not sensitive to the reactants and reaction conditions.

Solvent Evaporation

Microcapsules can be prepared by removing a volatile solvent from an emulsion of two immiscible liquids, e.g., an oil-in-water, oil-in-oil, or water-in-oil-in-water emulsion. The material that forms the shell is soluble in the volatile solvent. The filler material is dissolved, dispersed, or emulsified in the solution. Suitable solvents include methylene chloride and ethyl acetate. Solvent evaporation is a preferred method for encapsulating water soluble filler materials, for example, polypeptides. When such water-soluble components are to be encapsulated, a thickening agent is typically added to the aqueous phase, then the solution is cooled to gel the aqueous phase before the solvent is removed. Dispersing agents can also be added to the emulsion prior to solvent removal. Solvent is typically removed by evaporation at atmospheric or reduced pressure. Microcapsules of less than 1 μm in diameter or more than 1000 μm in diameter can be prepared using solvent evaporation methods.

Centrifugal Force Encapsulation

Microencapsulation by centrifugal force typically utilizes a perforated cup containing an emulsion of shell and filler material. The cup is immersed in an oil bath and spun at a fixed rate, whereby droplets including the shell and filler material form in the oil outside the spinning cup. The droplets are gelled by cooling to yield oil-loaded particles that can be subsequently dried. The microcapsules thus produced are generally relatively large. In another variation of centrifugal force encapsulation referred to as rotational suspension separation, a mixture of filler material particles and either molten shell or a solution of shell material is fed onto a rotating disk. Coated particles are flung off the edge of the disk, where they are gelled or desolvated and collected.

Submerged Nozzle Encapsulation

Microencapsulation by submerged nozzle generally involves spraying a liquid mixture of shell and filler material through a nozzle into a stream of carrier fluid. The resulting droplets are gelled and cooled. The microcapsules thus produced are generally relatively large.

Desolvation

In desolvation or extractive drying, a dispersion filler material in a concentrated shell material solution or dispersion is atomized into a desolvation solvent, typically a water-miscible alcohol when an aqueous dispersion is used. Water-soluble shell materials are typically used, including maltodextrins, sugars, gums, and the like. Preferred desolvation solvents include water-miscible alcohols such as 2-propanol, polyglycols, and the like. The resulting microcapsules do not have a distinct filler material phase. Microcapsules thus produced typically contain less than about 15 wt. % filler material, but in certain embodiments can contain more filler material.

Liposomes

Liposomes are microparticles typically ranging in size from less than about 30 nm to greater than 1 mm. They consist of a bilayer of phospholipid encapsulating an aqueous space. The lipid molecules arrange themselves by exposing their polar head groups toward the aqueous phase, and the hydrophobic hydrocarbon groups adhere together in the bilayer forming close concentric lipid leaflets separating aqueous regions. Medicaments can either be encapsulated in the aqueous space or entrapped between the lipid bilayers. Where the medicament is encapsulated depends upon its physiochemical characteristics and the composition of the lipid. Liposomes can slowly release any contained medicament through enzymatic hydrolysis of the lipid.

Miscellaneous Microencapsulation Processes

While the microencapsulation methods described above are generally preferred for preparing microcapsules for use in preferred embodiments, other suitable microencapsulation methods can also be used, as are known to those of skill in the art. Moreover, in certain embodiments, it can be desired to incorporate an unencapsulated medicament or other substance directly onto the chitosan substrate. Alternatively, the medicament or other substance can be incorporated into a solid matrix of a carrier substance, then deposited on the chitosan substrate. In such embodiments, since the medicament or other substance and the substrate will come into contact prior to coming in contact with the wound, the medicament or other substance is preferably not substantially sensitive to the substrate. The microcapsules that are deposited on the substrate can all be of the same type and contain the same medicaments or other substances, or can include a variety of types and/or encapsulated medicaments and/or other substances.

Preferred Microencapsulated Medicaments

In preferred embodiments, medicaments or other ingredients can be encapsulated into hydrophilic gelatin microcapsules prior to deposition on the chitosan substrate. Gatifloxacin is an especially preferred antibiotic that can be encapsulated and deposited on a hemostatic material so as to provide an effective sterilizing dosage of the antibiotic to the wound from the hemostatic material.

Another preferred embodiment employs hemostatic materials including hydrophilic gelatin microcapsules containing a chemotherapeutic agent. Such materials are particularly well suited for use in application to a tumor bed after surgery to both stop the oozing and gradually release the chemotherapy agent encapsulated therein.

Materials Comprising Hemostatic Agent Deposited on Hemostatic Support

The hemostatic agents of preferred embodiment are deposited on the hemostatic supports of preferred embodiments. The form of the hemostatic support will depend upon the application for which it is to be employed.

Hemostatic Puff

Hemostatic puffs are a particularly preferred form, wherein the substrate comprises a puff—a fibrous, cotton-like material that can be manipulated into a suitable shape or size so as to accommodate a particular wound configuration. In a preferred embodiment, a puff is prepared from chitosan fibers and microporous polysaccharide microspheres as follows. Chitosan fibers prepared according to conventional methods are manually or mechanically torn into pieces and the pieces are flattened and layered together. An acetic acid solution or other acidic solution (pH value preferably from about 3.0 to about 4.5) is sprayed onto a first layer as a wetting agent to control the surface moisture level of the chitosan fibers, thereby forming a sticky surface on which to fix the microporous polysaccharide microspheres. The microporous polysaccharide microspheres are sprayed or otherwise deposited onto the first chitosan fiber layer, and then another layer of chitosan is placed on top. The deposition process (acidic solution followed by deposition of microporous polysaccharide microspheres) is then repeated and the layers built up to a desired level. A preferred thickness for the fabric can be obtained by selecting the total number of layers. Microporous polysaccharide microspheres are preferably added to the fiber layers in a quantity sufficient to yield a puff comprising up to about 50% by weight of microporous polysaccharide microspheres. The resulting hemostatic material is dried, optionally in an oven and optionally under vacuum, to yield a hemostatic puff.

While it is generally preferred to employ an acetic acid solution, other acidic solutions of similar pH can also be preferably employed. In certain-embodiments, it can be preferred to employ a solution that is not acidic. In such embodiments, another suitable material in suitable form that provides adhesion between chitosan fibers and the microporous polysaccharide microspheres can be employed, for example, gelatin, starch, carageenan, guar gum, collagen, pectin, and the like. While chitosan is a preferred substrate for preparing a hemostatic puff, other fibrous substrates, particularly fibrous polysaccharide substrates, are also suitable for use.

By adjusting the moisture level in the chitosan fibers, the hemostatic agent loading capacity of the fibers can be optimized. The liquid assists in adhering the fibers and microparticles to each other. It can also be possible to increase the loading capacity by employing thinner fibers. The fibers can be of uniform thickness, or comprise a mixture of thicknesses. Thinner fibers can also adhere more firmly to an artery, vein, or other wound.

In preparing a hemostatic puff, e.g., a puff comprising microporous polysaccharide microsphere-loaded chitosan fibers, it is generally preferred that the resulting puff contain from about 1.0 wt. % or less to about 60 wt. % or more microporous polysaccharide microspheres or other hemostatic agent, more preferably from about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 wt. % to about 45, 50, or 55 wt %. In certain embodiments, however, higher or lower levels of microporous polysaccharide microspheres can be preferred. If a different hemostatic agent is employed, or other components are to be added to the chitosan fibers or other fibrous substrate, different loading levels can be preferred.

Hemostatic Fabric

Hemostatic fabric can be prepared from chitosan fibers and microporous polysaccharide microspheres according to the method described above for preparation of hemostatic puffs, with the following modifications. Microporous polysaccharide microspheres are preferably added to the fiber layers in a quantity sufficient to yield a fabric comprising from about 20 wt. % or less to 50 wt. % microporous polysaccharide microspheres. The layers are pressed flat and dried, preferably with heat and preferably under vacuum. It is generally preferred that one side of the fabric has a smooth surface and the other side of the fabric have a rough surface (e.g., in the case of chitosan and microporous polysaccharide microspheres, a TEFLON™ surface applied to a surface during heating yields a smooth side, while a release paper applied to a surface yields a rough surface). In preferred embodiments, the rough surface is exposed to the wound so as to maximize contact of the microporous polysaccharide microsphere-loaded chitosan fibers with the wound, resulting in an improved hemostatic effect and superior adherence to the wound.

In preparing a hemostatic fabric, e.g., a fabric comprising microporous polysaccharide microsphere-loaded chitosan fabric, it is generally preferred that the resulting fabric contain from about 1.0 wt. % or less to about 95 wt. % or more microporous polysaccharide microspheres or other hemostatic agent, more preferably from about 2.0, 3.0, 4.0, 5.0, 6.0, 7.0, 8.0, or 9.0 wt. % to about 60, 65, 70, 75, 80, 85, or 90 wt. %, and most preferably from about 10, 11, 12, 13, 14, 15, 16, 17, 18, 19, 20, 21, 22, 23, 24, or 25 wt. % to about 25, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wt. %. In certain embodiments, however, higher or lower levels of microporous polysaccharide microspheres can be preferred. If a different hemostatic agent is employed, or other components are to be added to the fabric, different loading levels can be preferred.

The hemostatic fabric can be provided in the form of a sheet of a pre-selected size. Alternatively, a larger sheet of hemostatic fabric can be cut or trimmed to provide a size and shape appropriate to the wound. Although the hemostatic fabric is bioabsorbable, in cutaneous or topical applications it is preferably removed from the wound after a satisfactory degree of hemostasis is achieved. When the hemostatic fabric is employed in internal applications, it is preferably left in place to be absorbed by the body over time. Such hemostatic fabrics are particularly well suited for use in the treatment of oozing wounds.

It is generally preferred to employ a nonwoven hemostatic fabric. However, in certain embodiments in can be preferred to employ a woven hemostatic fabric. The fabric can include one or more layers, preferably 2, 3, 4, 5, 6, 7, 8, or 9 layers to about 10, 15, 20, or 25 layers or more, and can include all woven layers, all nonwoven layers, or a combination of woven and nonwoven layers.

Hemostatic Sponge

A hemostatic sponge can be prepared according to methods known in the art for preparing a porous sponge from a biocompatible or bioabsorbable polymeric material, e.g., chitosan. Such methods typically involve preparation of a solution of the polymeric material, crosslinking agents, and foaming agents. The sponge can be loaded with hemostatic agent at any convenient point or points in the process, e.g., during formation of the sponge, or after preparation of the sponge.

In preparing a hemostatic sponge, it is generally preferred that the resulting sponge contain from about 1.0 wt. % or less to about 95 wt. % or more microporous polysaccharide microspheres or other hemostatic agent, more preferably from about 2.0, 3.0, 4.0, 5.0, 6.0, 7.0, 8.0, 9.0, or 10.0 wt. % to about 60, 65, 70, 75, 80, 85, or 90 wt. %, and most preferably from about 11, 12, 13, 14, 15, 16, 17, 18, 19, 20, 21, 22, 23, 24, or 25 wt. % to about 30, 31, 32, 33, 34, 35, 36, 37, 38, 39, 40, 41, 42, 43, 44, 45, 46, 47, 48, 4, 50, 51, 52, 53, 54, or 55 wt. %. In certain embodiments, however, higher or lower levels of microporous polysaccharide microspheres can be preferred. If a different hemostatic agent is employed, or other components are to be added to the sponge, different loading levels can be preferred.

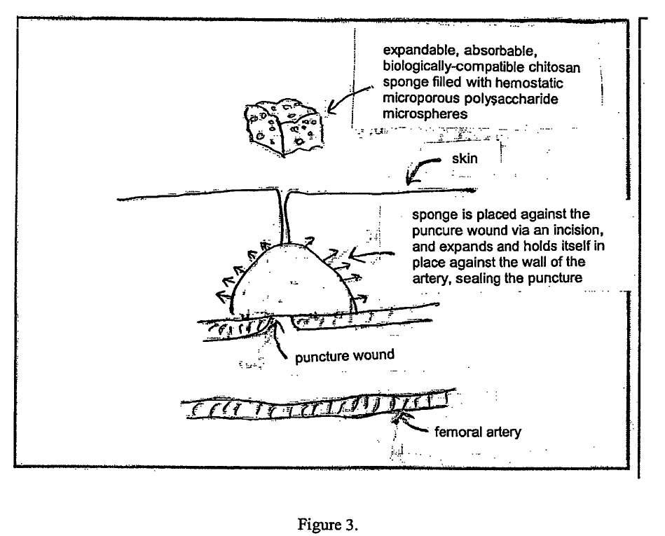

FIG. 3 depicts sealing a femoral artery puncture with a hemostatic sponge. The expandable, absorbable, biologically-compatible chitosan sponge filled with hemostatic microporous polysaccharide microspheres is placed against the puncture wound via an incision in the skin. The hemostatic sponge expands and holds itself in place against the wall of the artery, sealing the puncture.

Hemostatic Sutures

The hemostatic substrates of preferred embodiments can be fabricated into sutures. In a preferred embodiment, chitosan fibers or fibers of other materials are fabricated into microsutures upon which the hemostatic agent is deposited. Processes for suture fabrication include extrusion, melt spinning, braiding, and many others. The synthesis of raw suture materials is accomplished by any number of processes within the textile industry. Suture sizes are given by a number representing the diameter ranging in descending order from 10 to 1 and then 1-0 to 12-0, with 10 being the largest and 12-0 being the smallest.

Sutures can comprise monofilaments or many filaments twisted together, spun together, or braided. The sutures of preferred embodiments exhibit satisfactory properties including stress-strain relationship, tensile strength, rate of retention, flexibility, intrinsic viscosity, wettability, surface morphology, degradation, thermal properties, contact angle of knots, and elasticity. The sutures can comprise filaments of the same material, or filaments comprised of different materials.

In preparing a hemostatic suture, it is generally preferred that the resulting suture contain from about 1.0 wt. % or less to about 95 wt. % or more microporous polysaccharide microspheres or other hemostatic agent, more preferably from about 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, or 29 wt. % to about 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, or 90 wt. %. In certain embodiments, however, higher or lower levels of microporous polysaccharide microspheres can be preferred. If a different hemostatic agent is employed, or other components are to be added to the suture, different loading levels can be preferred.

Because of the hemostatic nature of the sutures of preferred embodiments, they are not suitable for blood vessel anastomosis.

Hemostatic Powders

The hemostatic substrates of preferred embodiments can be formed into a powder and mixed with the hemostatic agent. For example, chitosan particles can be combined with a hemostatic agent such as microporous polysaccharide microspheres. Such hemostatic powders can be employed as a void filler following tooth extraction.

In preparing a hemostatic powder, it is generally preferred that the resulting powder contain from about 1.0 wt. % or less to about 95 wt. % or more microporous polysaccharide microspheres or other hemostatic agent, more preferably from about 2, 3, 4, 5, 6, 7, 8, 9, 10, 11, 12, 13, 14, 15, 16, 17, 18, 19, 20, 21, 22, 23, 24, 25, 26, 27, 28, or 29 wt. % to about 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, or 90 wt. %. In certain embodiments, however, higher or lower levels of microporous polysaccharide microspheres can be preferred. If a different hemostatic agent is employed, or other components are to be added to the powder, different loading levels can be preferred.

Hemostatic Matrices

Three-dimensional porous matrices can be prepared from sintered polymer particles, for example, chitosan particles, and the hemostatic agent infused into the pores. Alternatively, microcapsules comprising a chitosan shell encapsulating a hemostatic agent can be sintered to form a matrix.

In preparing a hemostatic matrix, it is generally preferred that the resulting matrix contain from about 1.0 wt. % or less to about 95 wt. % or more microporous polysaccharide microspheres or other hemostatic agent, more preferably from about 2, 3, 4, 5, 6, 7, 8, 9, 10, 11, 12, 13, 14, 15, 16, 17, 18, 19, 20, 21, 22, 23, 24, 25, 26, 27, 28, or 29 wt. % to about 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, or 90 wt. %. In certain embodiments, however, higher or lower levels of microporous polysaccharide microspheres can be preferred. If a different hemostatic agent is employed, or other components are to be added to the matrix, different loading levels can be preferred.

Wound Dressings

While it is generally preferred to apply the hemostatic material (for example, a hemostatic fabric, sponge, puff, matrix, or powder prepared as described above, or another form) directly to the wound, in certain embodiments it can be preferred to incorporate the hemostatic material into a wound dressing including other components.

To ensure that the hemostatic material remains affixed to the wound, a suitable adhesive can be employed, for example, along the edges or a side of the hemostatic fabric, sponge or puff. Although any adhesive suitable for forming a bond with skin or other tissue can be used, it is generally preferred to use a pressure sensitive adhesive. Pressure sensitive adhesives are generally defined as adhesives that adhere to a substrate when a light pressure is applied but leave no residue when removed. Pressure sensitive adhesives include, but are not limited to, solvent in solution adhesives, hot melt adhesives, aqueous emulsion adhesives, calenderable adhesive, and radiation curable adhesives. Solution adhesives are preferred for most uses because of their ease of application and versatility. Hot melt adhesives are typically based on resin-tackified block copolymers. Aqueous emulsion adhesives include those prepared using acrylic copolymers, butadiene styrene copolymers, and natural rubber latex. Radiation curable adhesives typically consist of acrylic oligomers and monomers, which cure to form a pressure sensitive adhesive upon exposure to ultraviolet lights.

The most commonly used elastomers in pressure sensitive adhesives include natural rubbers, styrene-butadiene latexes, polyisobutylene, butyl rubbers, acrylics, and silicones. In preferred embodiments, acrylic polymer or silicone based pressure sensitive adhesives are used. Acrylic polymers generally have a low level of allergenicity, are cleanly removable from skin, possess a low odor, and exhibit low rates of mechanical and chemical irritation. Medical grade silicone pressure sensitive adhesives are preferred for their biocompatibility.

Amongst the factors that influence the suitability for a pressure sensitive adhesive for use in wound dressings of preferred embodiments are the absence of skin irritating components, sufficient cohesive strength such that the adhesive can be cleanly removed from the skin, ability to accommodate skin movement without excessive mechanical skin irritation, and good resistance to body fluids.

In preferred embodiments, the pressure sensitive adhesive comprises a butyl acrylate. While butyl acrylate pressure sensitive adhesives are generally preferred for many applications, any pressure sensitive adhesive suitable for bonding skin can be used. Such pressure sensitive adhesives are well known in the art.

As discussed above, the hemostatic materials of preferred embodiments generally exhibit good adherence to wounds such that an adhesive, for example, a pressure sensitive adhesive, is generally not necessary. However, for ease of use and to ensure that the hemostatic material remains in a fixed position after application to the wound, it can be preferable to employ a pressure sensitive adhesive.

While the hemostatic puffs, fabrics and other hemostatic materials of preferred embodiments generally exhibit good mechanical strength and wound protection, in certain embodiments it can be preferred to employ a backing or other material on one side of the hemostatic material. For example, a composite including two or more layers can be prepared, wherein one of the layers is the hemostatic material and another layer is, e.g., an elastomeric layer, gauze, vapor-permeable film, waterproof film, a woven or nonwoven fabric, a mesh, or the like. The layers can then be bonded using any suitable method, e.g., adhesives such as pressure sensitive adhesives, hot melt adhesives, curable adhesives, application of heat or pressure such as in lamination, physical attachment through the use of stitching, studs, other fasteners, or the like.

Other components can be combined with the hemostatic materials for use in wound dressings as are known in the art, such as preservatives, stabilizers, dyes, buffers, alginate pastes or beads, hydrocolloid pastes or beads, hydrogel pastes or beads, as well as medicaments and other therapeutic agents as described above.

Interaction between Chitosan Substrate and Microporous Polysaccharide Microspheres

Both chitosan and microporous polysaccharide microspheres exhibit a degree of hemostatic effect, but when combined yield an unexpectedly superior hemostatic material that exhibits surprising effectiveness in promoting hemostasis.

The literature suggests that the hemostatic effect of chitosan may not follow the coagulation cascade pathways as described above, because chitosan can still cause coagulation of blood from which all of the platelets, white blood cells, and plasma have been removed. Chitosan's hemostatic effect is most likely due to its ability to cause erythrocytes to coalesce with each other, thereby forming a blood clot. When chitosan fibers come into contact with blood, the blood penetrates into the network formed by chitosan fibers. Chitosan is hydrophilic and is wettable to form a hydrogel, which may assist in adhering the fibers to the wound. Another hypothesis is that chitosan, a naturally positively charged polysaccharide, can interact with negative charges on the surface of blood proteins to cause erythrocytes to coalesce with each other.

Both microporous polysaccharide microspheres and chitosan are hydrophilic and biodegradable. They have a similar biocompatibility and a similar hemostatic mechanism. They are also easily and effectively combined with each other and exhibit strong physical adsorption to each other. The strong physical adsorption between microporous polysaccharide microspheres and chitosan is believed to be due, at least in part, to the similarity in their skeletal chemical structures, both of which are based on glucose units. Both microporous polysaccharide microspheres and chitosan have strong affinity to cells as well as to each other, thereby resulting in a surprisingly effective hemostatic material when combined.

The loading efficiency of microporous polysaccharide microspheres in a puff comprising chitosan fibers was determined. Loading efficiencies of up to 90% can be achieved while maintaining the pliability of the puff. At loading efficiencies above 90%, hardening of the puff can result, but can be acceptable in certain embodiments.

The expansion of microporous polysaccharide microspheres and chitosan after they contact water was measured. It was observed that pure microporous polysaccharide microspheres absorb water and expand to generate pressure against surrounding structures. However, no clinically significant expansion of microporous polysaccharide microspheres deposited on a chitosan fiber puff was observed upon contact with water. The measurements were conducted as follows: 19 g of TRAUMADEX™ microporous polysaccharide microspheres were placed in a device, the diameter of which was 1.55 cm, to measure expansion. Water was added to the TRAUMADEX™, resulting in the water's adsorption. Weight was added to the top of the device to prevent TRAUMADEX™ from expanding. The weight added corresponds to the pressure that TRAUMADEX™ produced after it contacted water. In the experiment, the difference in the weight applied before contact of the TRAUMADEX™ with water and the weight applied after contact of the TRAUMADEX™ with water was 270 g. Accordingly, the pressure which TRAUMADEX™ exerted after it contacted water was 107 mm Hg. The same method was employed to measure the expansion of TRAUMADEX™ deposited on a chitosan puff, but the volume change observed was too small to be measured. It is believed that the porous chitosan puff provides sufficient space for the expanded TRAUMADEX™ such that no significant volume change of the TRAUMADEX™ deposited on the chitosan puff can be detected upon contact with water.

Closure of Femoral Artery Puncture Wounds

A hemostatic puff comprising TRAUMADEX™ deposited on chitosan fibers was developed for use in conjunction with a femoral artery puncture wound closure device. The hemostatic puff, is wrapped around the blood indication catheter of the wound closure device, and can be efficiently and effectively delivered to the top of the puncture wound. In a particularly preferred embodiment, both the hemostatic puff and an adhesive suitable for securing the puff to the wound are delivered by the wound closure device. A vascular wound closure device suitable for use with the hemostatic puffs of preferred embodiments is disclosed in U.S. application Ser. No. 10/463,754 filed Jun. 16, 2003 and entitled “VASCULAR WOUND CLOSURE DEVICE AND METHOD”, the contents of which are hereby incorporated by reference in their entirety.

In a venous laceration, the conventional method of repairing the laceration involves temporarily stopping the bleeding, occluding the vein, suctioning out the blood, then suturing or clipping the laceration to repair it. A vessel patch can also be required in conventional methods. The hemostatic fabrics of preferred embodiments can also be employed to treat venous or arterial lacerations merely by compressing the fabric to the laceration and allowing it to remain in place and eventually be absorbed by the body.

Preparation of Chitosan

Chitin is present in crustacean shells as a composite with proteins and calcium salts. Chitin is produced by removing calcium carbonate and protein from these shells, and chitosan is produced by deacetylation of chitin in a strong alkali solution. U.S. Pat. No. 3,533,940, the contents of which are incorporated by reference herein in its entirety, describes a method for the preparation of chitosan. Chitin can be derived from crab, crayfish, shrimp, prawn, and lobster shells, as well as from the exoskeletons of marine zooplankton, including coral and jellyfish. Insects, such as butterflies and ladybugs, can have chitin in their wings, and the cell walls of yeast, mushrooms and other fungi can also contain chitin. In addition to natural sources, synthetically produced chitin and/or chitosan is also suitable for use in preferred embodiments.

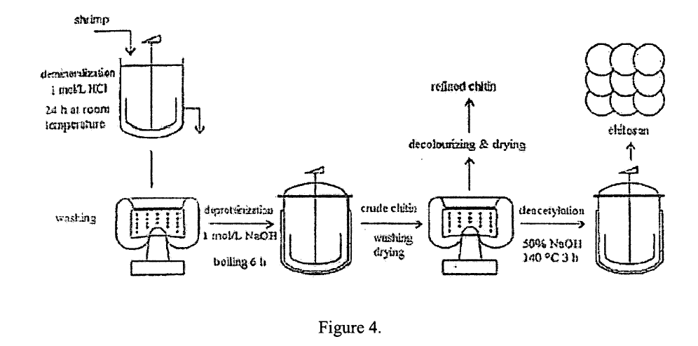

A preferred method for obtaining chitosan from crustacean shells is as follows. Calcium carbonate is removed by immersing the shell in dilute hydrochloric acid at room temperature for 24 hours (demineralization). Proteins are then extracted from the decalcified shells by boiling them with dilute aqueous sodium hydroxide for six hours (deproteinization). The demineralization and deproteinization steps are preferably repeated at least two times to remove substantially all of the inorganic materials and proteins from the crustacean shells. The crude chitin thus obtained is washed then dried. The chitin is heated at 140° C. in a strong alkali solution (50 wt. %) for 3 hours. Highly deacetylated chitosan exhibiting no significant degradation of the molecular chain is then obtained by intermittently washing the intermediate product in water preferably two or more times during the alkali treatment. FIG. 4 schematically depicts a process for obtaining chitosan from shrimp waste.

Preparation of Chitosan Fiber

In a preferred embodiment, a wet spinning method is employed to prepare chitosan fiber. First, chitosan is dissolved in a suitable solvent to yield a primary spinning solution. Preferred solvents include acidic solutions, for example, solutions containing trichloroacetic acetic acid, acetic acid, lactic acid, and the like. However any suitable solvent can be employed. The primary spinning solution is filtered and deaerated, after which it is sprayed under pressure into a solidifying bath through the pores of a spinning jet. Solid chitosan fibers are recovered from the solidified bath. The fibers can be subjected to further processing steps, including but not limited to drawing, washing, drying, post treatment, functionalization, and the like.

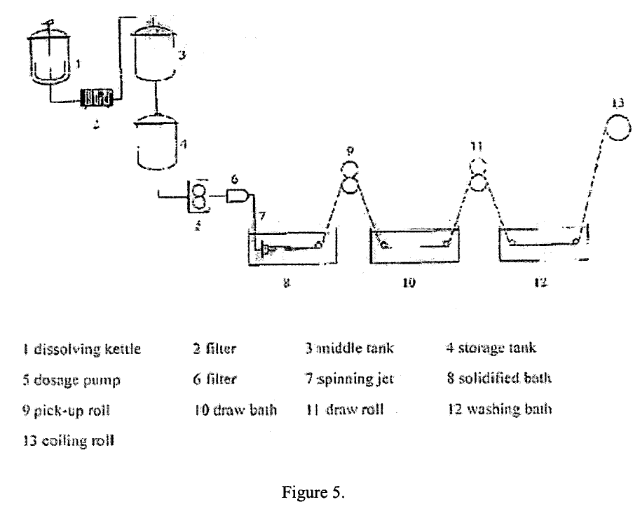

A preferred method for preparing chitosan fiber suitable for fabrication into the hemostatic materials of preferred embodiments is as follows. The primary chitosan spinning solution is prepared by dissolving 3 parts chitosan powder in a mixed solvent containing 50 parts trichloroacetic acid (TDA) to 50 parts methylene dichloride at a solvent temperature of 5° C. The resulting primary spinning solution is filtered and then deaerated under vacuum. A first solidifying bath comprising acetone at 14° C. is employed. The aperture of the spinning jet is 0.08 mm, the hole count is forty-eight, and the spinning velocity is 10 m/min. The spinning solution is maintained at 20° C. by heating with recycled hot water. The chitosan fibers from the acetone bath are recovered and conveyed via a conveyor belt to a second solidifying bath comprising methanol at 15° C. The fibers are maintained in the second solidifying bath for ten minutes. The fibers are recovered and then coiled at a velocity of 9 m/min. The coiled fibers re neutralized in a 0.3 g/l KOH solution for one hour, and then washed with deionized water. The resulting chitosan fiber is then dried, after which it is ready for fabrication into the hemostatic materials of preferred embodiments. FIG. 5 schematically depicts an apparatus for preparing chitosan fibers.

### Experiments

Preparation of Chitosan Puff

A hemostatic puff was prepared from chitosan fibers as follows. The chitosan fiber was laid layer by layer. Hemostatic powder (TRAUMADEX™) was sprayed onto each layer, along with an acetic acid solution which functioned to glue hemostatic powder to chitosan fibers. After drying under vacuum, the hemostatic puff was obtained.

First, a glue solution was prepared comprising acetic acid solution with pH value of from 3.0 to 4.5. The chitosan fibers were torn into pieces. After laying down a first layer of such chitosan pieces, the acetic acid solution was sprayed onto the chitosan pieces, and then the hemostatic powder was added. A second layer was formed upon the first layer by the same procedure. Layers were built up in this fashion until 5-10 layers were obtained. The more layers that were built up, the more homogeneous was the distribution of hemostatic powder. The acetic acid solution acted not only as a glue between the hemostatic powder and chitoan fibers, but also between chitosan layers. The hemostatic powder loading efficiency is provided in Table 1.

TABLE 1
Drug Loading Efficiency of Chitosan (CS) Puff

| CS weight (g) | | | | |
|---|---|---|---|---|
| after drying/ | Drug | CS + drug | Loading | Fiber |
| before drying | (g) | (after drying) (g) | Efficiency | Condition |
| 1.96/(2.19) | 0 | 1.96 | — | loose/flexible |
| 1.92/(2.15) | 0.25 | 2.15 | 92.0% | loose/flexible |
| 1.82/(2.03) | 0.51 | 2.28 | 90.1% | loose/flexible |
| 1.98/(2.21)* | 1.01 | 2.96 | 97.0% | hard |

*Two times as much water was sprayed onto the fibers compared to that used in the other examples.

This hemostatic chitosan puff thus prepared exhibited good hemostatic function and swelling ability. When placed on or in a wound, the puff absorbed the blood immediately. The blood passed through the first few chitosan layers, then immediately solidified to prevent further bleeding. This hemostatic chitosan puff biodegrades to nontoxic materials in the body after a period time, thus surgery is not needed to remove the puff if it is placed internally.

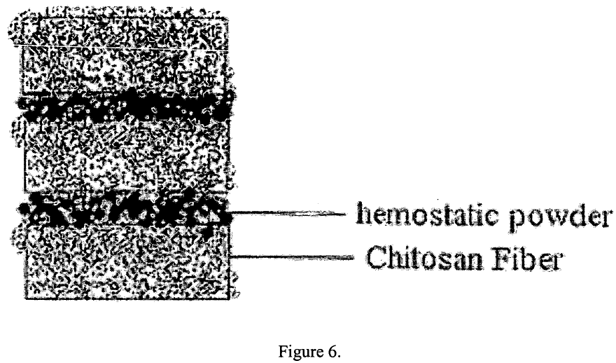

FIG. 6 schematically depicts a layered hemostatic material comprising alternate layers of chitosan fiber and hemostatic powder.

Estimation of the Expansion of TRAUMADEX™ Powder

The expansion of TRAUMADEX™ hemostatic powder was estimated. Hemostatic powder expands upon absorption of water, resulting in an exertion of pressure. Upon expansion, weight was added to maintain a pressure balance to maintain a constant volume of hemostatic powder. The maximum weight correlates to the maximum pressure that the hemostatic powder produced upon expansion, which was converted to intensity of pressure. At the beginning of the experiment, pre-weighted hemostatic powder was added to an injector and its volume marked with a red line. Then, an amount of water was added to the injector via a burette. To counteract the pressure created by the water, weight was added on the top of the injector. The weight that was added to counteract pressure produced by water adsorption by the hemostatic powder was identified as W₀. To keep the volume constant as water was absorbed, more weight was added. The total weight after absorption was complete was identified as Wₜ. The value of Wₜ−W₀ corresponded to the pressure produced by the expansion of the hemostatic powder. While not precise, the experiment provided a semi-quantitative result that enabled comparisons between materials to be made.

The diameter of the injector employed was 1.55 cm, and 1 g of hemostatic powder was placed in the injector. The value of Wₜ−W₀ was 270 g, corresponding to a pressure of 107 mmHg. It was attempted to measure the expansion of a hemostatic puff, but the volume change was too small to be measured.

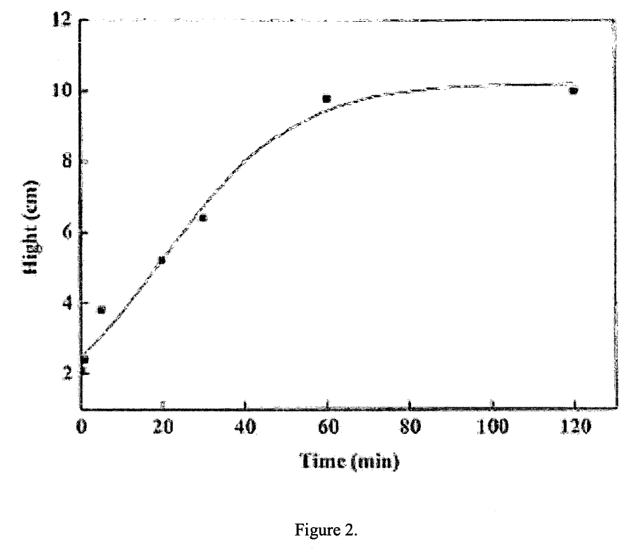

The expanding ability of the hemostatic agent and hemostatic cotton in an open condition was also characterized. First, 1.0 g hemostatic powder was added to a measuring cylinder. The initial volume of hemostatic powder was measured as V₀. Then, 10.0 g water was added to the measuring cylinder, and after passage of a predetermined time interval the hemostatic powder volume was measured (Vₜ). FIG. 2 illustrates the volume change of hemostatic powder at different time intervals. Hemostatic powder was observed to adsorb much water and expand. However, the mechanical strength of this expanded hemostatic powder was very poor, and displayed as paste.

Preparation of Chitosan Fabric

Hemostatic fabric was prepared according to the following procedure. First, an aqueous solution of 1 wt. % acetic acid with a pH of 3.0 was prepared. Chitosan fiber was separated into pieces and homogeneously laid on a glass plate covered with releasing paper to form a thin layer. The aqueous acetic acid solution was sprayed onto the chitosan fiber surface, and a specified amount of hemostatic powder was distributed over the chitosan fiber. Additional layers were built up by the same procedure. After a predetermined amount of aqueous acetic acid solution was sprayed onto the uppermost chitosan fiber layer, a flat plate of polytetrafluoroethylene (TEFLON™) was placed on the uppermost chitosan fiber layer. Samples comprising five layers were thus prepared.

The layers were compressed and the entire system was placed in a vacuum oven and dried under vacuum for three hours at 50° C. while maintaining the compression. The TEFLON™ plate and releasing paper were removed, and the non-woven hemostatic fabric was recovered. The upper layer which was in contact with the TEFLON™ plate was covered with a thin membrane of Chitosan, and the bottom layer which was in contact with the releasing paper was made up of nonwoven fibrous chitosan having a rough surface.

Animal Hemostatic Testing of the Chitosan-MPM (Microporous Polysaccharide Microsphere) Fleece and Fabric

Hemostatic tests were performed on injured large vessels (catheterized canine femoral artery) under heparinization, on swine femoral arteries, and on punctured rat femoral arteries and veins.

Catheterized Canine Femoral Artery

The model for control of brisk bleeding after arterial puncture and catheterization is the 3-4 canine femoral artery in heparinized animals. Three animals had an 11.5 French catheter placed for 4-6 hours in the femoral artery, were heparinized with activated clotting times (ACT) 2-3 times normal, and were maintained at normotensive levels by IV (intravenous) fluid replacement. The indwelling arterial catheter was removed and the chitosan-MPM patch (2×2 cm) immediately applied to the bleeding vessel with minimal pressure for 10 minutes. Videotapes documented these studies.

Dog Three—dog weight: 25.7 kg; sex F; coagulation time ACT 277 seconds. The catheter in the dog femoral artery was 11.5 F. 1-2 cm³ of chitosan-MPM was placed on the femoral artery puncture hole immediately after 11.5 F catheter was removed. Manual pressure was applied on the fleece for 10 minutes and bleeding was completely stopped with absolute hemostasis. Chitosan-MPM was applied to a femoral vein puncture hole, another 11.5 F catheter was removed, and held with manual pressure for 7 minutes. Complete hemostasis was achieved. Venous pressure was increased by proximal ligation and the chitosan-MPM adhered without bleeding.

Dog Four—dog weight: 25.4 kg; sex F; coagulation time ACT 280 seconds. 1-2 cm³ chitosan-MPM was placed on the femoral artery puncture hole immediately after 11.5 F catheter was removed with manual pressure for 10 minutes. Complete hemostasis with marked adherence of the fleece was noted.

Dog Five—dog weight: 23.1 kg; sex M; coagulation time, ACT 340 seconds. PVA treated chitosan-MPM fleece (1 cm³) was applied to the femoral artery puncture hole after the 11.5 F catheter was removed and manual pressure was applied for 10 minutes. Bleeding stopped but 30 seconds later, moderate bleeding from the puncture wound was noted. A second attempt using the same PVA treated chitosan-MPM fleece (10 minutes manual compression) failed. Chitosan-MPM non-woven fabric without PVA was then used to replace the relatively non-adherent PVA treated chitosan-MPM fleece. Complete hemostasis was achieved after 15 minutes of manual compression. The wound was observed for 20 minutes and no bleeding was noted. The non-PVA treated chitosan-MPM fabric adhered tightly to the artery and surrounding tissue. Artery with fabric was removed for pathology.

The dog experiments demonstrate that chitosan-MPM fleece (non-PVA treated) was remarkably effective as a hemostatic agent in the heparinized canine arterial catheterization model. A large bore catheter (11.5 F), left in place for 4-6 hours resulted in a significant molded, vascular breech and in the face of significantly prolonged coagulation time represented a real hemostatic challenge. Chitosan-MPM fleece also conformed to the arterial contour, did not interfere with distal flow, and was remarkably adherent. Chitosan-MPM fleece was equally effective in achieving hemostasis in the catheterized femoral vein and also remarkably adherent without interfering with flow. Chitosan-MPM fleece (PVA treated) achieved moderate to minimal hemostasis in one trial, and was relatively non-adherent. Complete hemostasis was secured with a non-PVA treated chitosan-MPM fabric patch.

Rat Punctured Femoral Artery and Vein

Femoral arteries and veins of 3 rats (OD 1.5 to 2 mm) were exposed bilaterally after barbiturate anesthesia achieved. Puncture wounds were made in each artery with a 30 gauge needle, and a pledget (3 mm³) of either chitosan-MPM fleece or fabric placed on the puncture site for 10 seconds and monitored for bleeding PVA treated material was not used. Control of bleeding from injured thin walled (100 min) rat femoral vessels is a hemostatic challenge. After exposing both femoral arteries, a 30 gauge needle was used to puncture the arteries to create an arterial laceration and brisk bleeding.

Rat No. 1—male, 520 g. The right femoral artery puncture wound was treated with a pledget of chitosan-MPM fabric. Gentle compression was applied to the pledget for 30 seconds, and after release there was very slight bleeding under the fabric. Gentle manual pressure was applied again for 10 seconds and the bleeding completely stopped. After 20 minutes observation of complete hemostasis, both proximal and distal ends of the femoral artery were ligated and a burst strength test was conducted. The fabric repaired wound remained intact at 120 mm Hg.

Rat No. 2—male, 525 g. The left femoral artery puncture wound was treated with a 3 mm² pledget of chitosan-MPM fabric. Manual compression was applied on the fabric for 10 seconds. After release of manual pressure there was slight bleeding under the fabric patch. 2 seconds of additional manual pressure was applied but minimal bleeding continued at a diminishing rate. No additional pressure was applied and bleeding stopped completely after 56 seconds. After 20 minutes of complete hemostasis, both proximal and distal end of the femoral artery were ligated and the burst strength test was conducted. Chitosan-MPM fabric repaired wound withstood arterial pressure until 300 mm Hg. The right femoral artery puncture wound was treated by placement of a fat pad over the injury. Manual compression was applied on the fatty tissue for 10 seconds. After release of the manual pressure there was profuse bleeding under the fatty tissue. No additional pressure was applied. The bleeding stopped after one minute and 27 seconds and 20 minutes later both proximal and distal end of the femoral artery were ligated and a burst strength test was conducted. The fatty tissue repaired wound failed at approximately 60 mm Hg.

Rat No 3—male 555 g. A right femoral artery puncture wound was treated with a chitosan-MPM 3 mm² pledget of mixed chitosan non-woven fabric, which was used to cover the wound. Manual compression was applied for 20 seconds, and after release complete hemostasis was secured. After 20 minutes of observation, both proximal and distal ends of the femoral artery were ligated and a burst strength test conducted. Chitosan-MPM patch withstood arterial pressure until 200 mm Hg. The right femoral artery puncture wound was covered with fatty tissue. Manual compression was applied on the fatty tissue for 20 seconds and after release of the manual pressure there was profuse bleeding. Bleeding stopped after one minute and 21 seconds with continued manual pressure. After that both proximal and distal ends of the femoral artery were ligated and a burst strength test was conducted, and the fatty tissue patch failed at less than 120 mm Hg (approx. 60).

The rat tests demonstrated that chitosan-MPM pledgets were remarkably effective in achieving complete hemostasis in the face of brisk bleeding from a puncture wound in a fragile vessel. The time required for the chitosan-MPM fabric to stop bleeding varied from 20 seconds to 56 seconds. Chitosan-MPM patch adheres very tightly to the vessel and can withstand high arterial pressures before failing. The rat femoral artery puncture model is an excellent screening system to study mechanisms for hemostasis and tissue adherence as well as screening of various chitosan-MPM formulations.

Swine Femoral Artery

Tests are conducted wherein a lethal large artery injury transects the femoral artery and femoral vein. The chitosan-MPM puff provides remarkable hemostasis in comparison to other methods that are utilized.

Chitosan-MPM Production Process

The term “chitosan” corresponds to a family of polymers that vary in degree of N-deacetylation (DA). Chitosan generally varies from about a 50 to 95% DA with variable viscosity, solubility, and hemostatic properties. Since the behavior of chitosan polymers, namely their reactivity, solubility, and ability to bind microporous polysaccharide microspheres, depends on the DA of chitin and chitosan, an assay to determine DA is desirable. The titration, FTIR spectroscopy and NMR spectroscopy are linked for chitosan assays. Prior to assay, all proteins and endotoxins are removed from the chitin as it is being produced for clinical application. Chitosan fibers are examined to determine their cross section, their tensile strength, breaking strength, loading strength, and their appearance. This industrial engineering process is utilized in the manufacture of chitosan fleece, chitosan sponge, as well as chitosan fabric. The amount of saturation of microporous polysaccharide microspheres is tested in model systems to determine appropriate physical characteristics for three major types of bleeding.

Characterizing the Structure and Properties of the Chitosan Fiber

Established and on-line methods for measuring the crystal structure, size, chitin DA, average molecular weight, content of heavy metals, and toxicity of chitosan fiber are used. Characterization includes fiber strength, pulling rate, mean fiber swelling as ratio of fiber diameter after absorption to that before absorption of distilled water, and pH. Chitosan having a DA of 50 to 95 wt. % is compared. Materials that are assayed include microporous polysaccharide microspheres, chitosan of varying DA, and chitosan-MPM. Measurements of water and blood absorption, rates of water and blood release, local retention (using gel strength), and screening tests for hemostasis are also conducted. Since erythrocyte polymerization (agglutination) is considered a major factor for chitosan induced blood coagulation, a simple hemagglutination test can be used for rapid screening of the product.

Simple hemagglutination assays are known in the art. Chitosan, chitosan-MPM, and microporous polysaccharide microspheres are prepared in stock solutions containing 2000 μg/ml. 10 fold dilutions are used to achieve final concentrations of 1000, 100, 10, and 0.1 μg/ml in a volume of 0.2 ml in 0.9% NaCl (normal saline). Human red cells (obtained from a blood bank) are rinsed twice with Alsever's Solution and twice with 0.9% sodium chloride. Sodium chloride is used to circumvent incompatibility between deacetylated chitin and other ions. Washed red cell are suspended in a saline solution (0.9% NaCl) and adjusted to 70% transmission with a colorimeter (Klett-Summerson, NO. 64 filter). An equal volume of red cell suspension (0.2 ml) is added to the various dilutions of chitosan-MPM, chitosan, and microporous polysaccharide microspheres. Tubes are incubated for 2 hours at room temperature before reading. Deacetylated chitin (chitosan) normally produces hemagglutination of human red blood cells at a concentration of 1 μg/ml.

Protein binding capacity can be determined using biomedical sensors utilizing reflectometry interference spectroscopy (RIFS), that enables the kinetics of the absorption of proteins onto the surface of chitosan, chitosan-MPM, and microporous polysaccharide microspheres alone to be determined. Once an optimal chitosan-MPM is reached for hemostasis, batches can be quickly evaluated for protein binding capacity and this parameter related to hemostatic effectiveness in the rat model cited above.

Optimization of microporous polysaccharide microspheres loading to chitosan can be achieved using systems other than the acetic acid treatment for loading microporous polysaccharide microspheres to the chitosan. For example, lactic acid is preferred for reduced toxicity when compared to acetic acid. The binding of microporous polysaccharide microspheres (a non-polar polysaccharide) to chitosan (a strongly cationic polysaccharide) can conceivably be enhanced by selective starch oxidations and generation of an anionic state.

Studies of the degradation kinetics of chitosan fibers, chitosan fleece, and fabric, both with and without microporous polysaccharide microspheres are conducted. Studies of the hemostatic mechanism of chitosan-MPM fleece and fabric are conducted using multi-photon imaging and spectroscopy to evaluate the molecular interaction of chitosan, chitosan-MPM, and microporous polysaccharide microspheres with human and porcine whole blood and platelets. These results are compared to the determinations offered by application of poly-N-acetyl glucosamine (p-GlcNAc or NAG). In vitro clot formation, red blood cell (RBC) aggregation, and platelet activation are studied.

Design and manufacture of a production line for large scale production of microporous polysaccharide microspheres mixed with chitosan fleece and chitosan non-woven fabric are conducted. Machines to perform the following functions are developed: loosening the chitosan fiber; carding the loosened fiber into a thin fleece net; moistening the chitosan fiber fleece by dilute acetic acid (or lactic acid) solution; homogeneously loading the microporous polysaccharide microspheres on the thin piece of moist chitosan fiber; rolling up the thin piece of the loaded chitosan fiber on a reel; and drying the fiber in a vacuum. A fully automatic or semi-automatic production line is designed and assembled to produce a standardized bulk quantity of chitosan-MPM fleece and nonwoven fabric. Tests of density of varied fleece preparations are conducted to optimize interstice size and optimal fleece density for hemostasis. Similar tests are performed on collagen fleece.

Optimizing Chitosan-MPM Formulations to Meet the Needs of Specific Hemorrhagic Diathesis

Formulations are optimized using models that the military has defined for testing and comparative evaluation of chitosan-MPM. These models include a fatal aortic punch lesion and large venous and diffuse capillary bleeding in a liver injury (swine). The model for remote closure of arterial catheterization lesions is taken from the literature and can be readily adapted to close lesions with chitosan-MPM. The oral bleeding model in the rabbit permits testing in a vascular organ system in an animal whose coagulation status can be readily modified (platelets, heparinization). This model has been tested with liquid chitosan as a hemostatic agent.

Fatal Aortic Injury Model in the Pig

This model was developed for hemostatic agent testing conducted at the U.S. Army Institute of Surgical Research, San Antonio, Tex. for the purpose of determining the optimal hemostatic dressing for high pressure arterial bleeding. The injury is a calibrated punch hole in the distal aorta of normotensive pigs. Nine different hemostatic dressings were evaluated for this otherwise 100% fatal injury. The only animals that lived 60 minutes received the American Red Cross Fibrin Dressing (Fibrin and Thrombin) or had suture repair of the lesion. All other hemostatic agents, including NAG, failed to control the aortal hemorrhage and no animals survived 60 minutes. Chitosan and microporous polysaccharide microspheres were not included in these experiments.

Five groups of five pigs (40 kg, immature Yorkshire cross swine male) are studied. One group is treated with American Red Cross Fibrin Dressing, the other four groups with chitosan fabric with or without microporous polysaccharide microspheres and chitosan fleece with or without microporous polysaccharide microspheres. Microporous polysaccharide microspheres alone generally do not control brisk arterial bleeding and are not included. Previous experiments demonstrated the fatality of the lesion untreated and that the animals can be rescued by suture repair. The objective of this study is to compare the American Red Cross Dressing to chitosan-based dressings. Survival, blood loss, and amount of IV resuscitable fluid to maintain normotension are determined.

Animals are premedicated (Telazol 4-6 mg/kg IM (intramuscular), Robinul 0.01 mg/kg IM), endotracheal anesthesia is maintained with 1-3% isofluorane and oxygen, and core temperature held between 37′-39° C. Indwelling arterial lines are placed for both proximal (carotid) and distal (femoral) MAP (Mean Arterial BP determinations) and a femoral IV line for resuscitative fluid administration. Pigs are spelenectomized, the spleen weighed, and replacement fluid (3×splenic weight of warm lactated Ringers) solution administered to correct for blood removal (spleen).

Hemodynamic stabilization is secured after splenectomy within 10 minutes and arterial blood samples (12 ml) are obtained prior to the aortic punch. The aortic injury is made immediately after aortic occlusion, and arterial blood is drawn 30 and 60 minutes after the injury. Prothrombin time, activated partial thromboplastin time, fibrinogen concentration, thromboelastogram, complete blood count, lactate and arterial blood gases are determined.

After the splenectomy and a 10 minute stabilization period, drains to continuous suction are positioned bilaterally in the lateral abdominal recesses. Rate of bleeding is determined by weighing the blood loss over time and is expressed as grams accumulated per 10 seconds. After cross-clamping the aorta above and below the site of the injury, (3 cm above the bifurcation of the distal aorta, aortotomy made with a 4.4 mm aortic hole punch) cross clamps are removed. Bleeding is initially tamponaded by placing a finger on the hole without vessel compression. At 0 time the finger relieves the tamponade and brisk arterial bleeding is allowed for 6 seconds. Blood is collected and rate of blood loss monitored by deflecting blood into the peritoneal cavity for drainage.

A polyethylene elastic sheet is placed between the dressing and gloved hand and after 6 seconds of brisk bleeding the test hemostatic dressing is applied for four minutes. Manual compression consists of complete aortal occlusion as manifested by a non-pulsatile femoral BP (MAP at 15 mm Hg). After 4 minutes, manual compression is relieved leaving the dressing and plastic sheet over the injury site. The injury site is observed for bleeding for 2 minutes. A key endpoint is a complete absence of bleeding after 2 minutes of observation. If bleeding persists, another 4 minutes of compression is administered. In the event of active bleeding or no hemostasis, resuscitation is discontinued and the animal allowed to die. In order to test the adherence of the test dressing with no evidence of bleeding, resuscitation is instituted with 37° C. lactated Ringer solution at a rate of 300 ml/min IV. A pre-aortotomy baseline MAP plus or minus 5 mm Hg is maintained for an additional 60 minutes. Death (a key endpoint) is a MAP<10 mm Hg and end tidal PCO2 less than 15 mm Hg. At the end of the experimental period (euthanasia at 1 hour in surviving animals) aortas are removed, opened, and evaluated. After the lesion is observed and photographed the size of the hole is measured to ensure uniformity of injury size, and the specimen fixed for histological examination to evaluate the hemostatic process (fibrin, platelets, extension into lumen).

Though the ARC hemostatic dressing has provided survival in this model it still has disadvantages. The “ideal” hemostatic dressing in addition to the parameters cited earlier controls large vessel arterial venous and soft tissue bleeding, adheres to the vessel injury but not to the glove or hands, is flexible, durable, and inexpensive, stable in an extreme environment, has a long shelf life, does not require mixing, poses no risk of disease transmission, does not require new training, and is manufactured from readily available materials. None of the dressings that have been tested or evaluated in the current setting meet all of these characteristics. The shortcoming of the fibrin-thrombin American Red Cross field dressing (ARC) is that it is fragile in its current form. The field dressing is stiff and thick when dry and some of the lyophilized material flakes off when the field dressing is grasped. The fibrin-thrombin dressing sticks to latex gloves and skin when wet. The handling characteristics of the chitosan fleece with microporous polysaccharide microspheres are superior to these prior art materials.

The Canine Femoral Artery Catheterization Model

This model has an extensive background literature for the evaluation of novel vascular sealing device. Femoral arteries are studied by percutaneous placement of standard vascular sheaths (7 French) with catheters inserted by the Seldinger technique. A total of 20 animals are utilized, 10 anticoagulated with IV heparin (150 units/kg) to activated clotting times (ACT) 3×normal. The ACT is measured just prior to insertion of the sealing device. Unheparinized animals have the contralateral femoral artery used as a control with only manual compression alone used to achieve hemostasis. Arterial sheaths and catheters are left in place for 1 hour to simulate an intervention duration. A vascular sealing device with the chitosan-microporous polysaccharide microspheres is used in one femoral artery and manual compression is utilized on the other femoral artery. Manual pressure applied to the puncture site is released and inspected every 5 minutes for the following key endpoints: external bleeding or hematoma formation, measurement of thigh circumference, integrity of the distal pedal pulses, and manual compression time required to achieve hemostasis. Animals are observed for an additional 90 minutes, then euthanized with an overdose of IV sodium pentobarbital and saturated potassium chloride. Prior to euthanization, animals are subjected to femoral angiography in each group.

A subgroup of animals survive with a follow-up examination at 2 weeks. This includes physical inspection of the arterial access, assessment of the distal pulses, femoral angiography, and histopathologic examination of the excised femoral artery puncture, site and surrounding tissue. Statistical analysis is expressed as mean standard deviation. The student's t-test unpaired is used for comparing the mean times to hemostasis within the different treatment groups. Preliminary animal studies are performed before proceeding to human clinical trials. Chitosan fleece with microporous polysaccharide microspheres and chitosan fabric with microporous polysaccharide microspheres both exhibit superior performance in controlling blood loss as well as the other parameters tested.

Model for Severe Large Venous Hemorrhage and Hepatic Injury (Swine)

This model has been extensively tested by the U.S. Army Combat Casualty Care Research Program. There is a large baseline of data regarding extent of injury and response to a variety of hemostatic agents. This data includes documentation of the extent of injury to large diameter veins, ability to apply hemostatic dressings in the face of massive bleeding, extent of blood loss, facility of instrumentation, lethality, and reproducibility of the experimental liver injury. Both the American Red Cross Hemostatic Dressing (ARC) and the experimental chitosan acetate sponge are effective hemostatic agents in this model. The hemostatic effectiveness of chitosan (fleece, fabric, with or without microporous polysaccharide microspheres) and the ARC dressing in the pig severe large venous hemorrhage model are tested.

The recommended conventional therapy for treating Grade V hepatic injuries (extensive parenchymal damage combined with major vascular lacerations) is tamponade with gauze sponges and later reoperation. The issues of biodegradability and wound healing have never been resolved with these hemostatic agents. Consequently, surviving animals are sacrificed one month post-injury to examine the healing wound and hemostatic agent degradation. Hemostatic control is monitored over a one-month period by weekly hepatic CT scans. Evidence of rehemorrhage requires intervention laparotomy and animal sacrifice. The post-injury and hemostatic repair course of the animals is monitored.

Crossbred commercial swine (males, 40-45 kg) are divided into 6 groups of 5 animals each. Test groups consist of gauze packing, ARC dressing, chitosan fleece with or without microporous polysaccharide microspheres, and chitosan fabric with or without microporous polysaccharide microspheres. Surgical preparation and anesthesia is as for the aortic punch injury model. Carotid artery and jugular vein lines are placed, and splenectomy and urinary bladder catheter placement is completed. Both hemodynamic (stable MAP for 15 minutes) and metabolic (rectal temperature 38-40° C., arterial blood pH 7.39-7.41) stabilization are achieved. Arterial blood samples are obtained. Each test animal must have a normal hematocrit, hemoglobin concentration, platelet count, prothrombin time, activated partial thromboplastin time, and plasma fibrinogen concentration to be included in the study. Drains are placed bilaterally (as in the aortotomy) for rate and quantitative blood loss calculation.

A liver injury is induced exactly as described in previous publications. Essentially, a specially designed clamp, “x” shaped, consisting of 4.5 cm sharpened tines and a base plate is used to make two penetrating liver lacerations. The standardized liver injuries are through-and-through stellate wounds, involving the left medial lobar vein, right medial lobar vein, portal hepatic vein, and hepatic parenchyma. 30 seconds after injury, warm (39° C.) lactated Ringers solution is started at a rate of 260 ml/min to restore the baseline MAP. The experimental hemostatic dressings are applied at the same time as IV fluids are initiated with manual compression via standardized applying pressure in a dorso-ventral direction. After one minute the wound is inspected for bleeding. If hemostasis is not complete, pressure is reapplied in the lateromedial direction. The sequence is repeated four times, with 60 seconds of compression.

The key endpoints of hemostasis are defined as the absence of any detectable bleeding from the injury. After application of the hemostatic treatment, the animals' abdomen is temporarily closed and the animal is observed for 60 minutes. The endpoint for death is a pulse of 0. Quantitative blood collection prior to treatment application is termed “pretreatment blood loss,” at the end of the study period—this is the “post-treatment blood loss.” Blood in the hemostatic agents is not included but total IV fluid replacement and estimated pre-injury blood volume is determined.

Adherence strength of the hemostatic dressing is estimated using the subjective scoring system reported by the military team who devised this protocol. Scores range from 1 to 5; 1=no adherence, 2=slight, 3=adherence to cause stretching of tissue in contact with hemostatic agent but not lifting liver from table, 4=adherence sufficient to partially lift liver from table, and 5=sufficient adherence to lift liver from table. The mean score for the 3 dressings from each animal is treated as a single value for adherence strength.

Key endpoints are survival, death, pretreatment blood loss, post-treatment blood loss, survival time, hemostasis at 1, 2, 3, and 4 minutes, and % resuscitation fluid volume. Key Parameters of injury are number of vessels lacerated correlated pre-treatment blood loss in ml and ml/kg body weight.

Chitosan fleece with microporous polysaccharide microspheres and chitosan fabric with microporous polysaccharide microspheres both exhibit superior performance in controlling blood loss as well as the other parameters tested.

Oral Bleeding Model: Lingual Hemostasis in the Rabbit

The oral bleeding model provides convenient hemostatic testing in a system with enhanced capillary blood flow (the tongue) and high fibrinolytic activity (oral mucosa). This model can easily have platelet function suppressed as well as be heparinized. The model has been used to evaluate the hemostatic effect of liquid chitosan in dilute acetic acid with the key endpoints of a reduced bleeding time after a standard incision. Descriptions of the model have been published and provide baseline data for results to be compared.

The hemostatic effectiveness of NAG, considered highly hemostatic for capillary hemorrhage, is compared with chitosan fleece with and without microporous polysaccharide microspheres and chitosan fabric with and without microporous polysaccharide microspheres. The key endpoints are lingual bleeding time, measured in minutes for the time the hemostatic agent is applied until hemostasis is complete. Rabbits are euthanized 1 to 14 days after the surgery and the lesions evaluated histologically. Rabbits with normal blood coagulation status, suppressed platelet activity, and heparin anticoagulation are studied.

New Zealand White (NZW) rabbits, 5-6 lbs, are studied for lingual hemostasis after using the model developed by Klokkevold, et al., consisting of a special metal stent sutured to the tongue in order to stabilize soft tissues and insure a consistent injury. Tongue incisions on the lateral border are made with a guarded 15 blade knife. Bleeding time measurements from the incision are made using the filter paper procedure of Coles. Blots are taken every 15 seconds until no blood staining occurs. Systemic bleeding and coagulation times are also determined. A total of 30 rabbits are studied, consisting of 6 groups of 5. The 6 groups consist of control (no treatment), NAG, chitosan fleece with or without microporous polysaccharide microspheres, and chitosan fabric with or without microporous polysaccharide microspheres. After animals are anesthetized (IM Ketamine HCl 35 mg/kg and Xylazine 5 mg/kg) an ocular speculum is inserted into the mouth to hold it open and the stainless steel stent sutured to the tongue to stabilize tissues. Tongue incisions are made with a depth of 2 mm, length 15 mm on the lateral border of the tongue with a guarded 15 blade. Incisions are immediately treated with the hemostatic agents and bleeding times measured. The method of tongue marking prior to incision is utilized to facilitate histologic sectioning post-marker.

The identical study as above in 30 rabbits, 5 groups of 5 each, is conducted in animals treated with the platelet function antagonist epoprostanol (prostacyclin or PGI₂). The protocol of Klokkevold is followed explicitly. Again, 30 rabbits are studied after the activated coagulation time has been prolonged 3× as well as increasing the mean systolic bleeding time by 40%. The histological exam includes SEM. Chitosan fleece with microporous polysaccharide microspheres and chitosan fabric with microporous polysaccharide microspheres both exhibit superior performance in controlling oral bleeding.

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

1. A hemostatic material, the material comprising a hemostatic agent and a therapeutic agent deposited on a hemostatic substrate, wherein the hemostatic substrate comprises chitosan.

2. The hemostatic material of claim 1, wherein the hemostatic agent comprises microporous polysaccharide microspheres.

3. The hemostatic material of claim 1, wherein the therapeutic agent comprises an anti-inflammatory agent.

4. The hemostatic material of claim 1, wherein the therapeutic agent comprises an anti-infective agent.

5. The hemostatic material of claim 1, wherein the therapeutic agent comprises an anesthetic.

6. The hemostatic material of claim 1, wherein the therapeutic agent comprises a chemotherapy agent.

7. The hemostatic material of claim 1, wherein the chitosan comprises a fiber.

8. The hemostatic material of claim 1, wherein the hemostatic material comprises from about 10 wt. % to about 50 wt. % of a hemostatic agent comprising microporous polysaccharide microspheres.

9. The hemostatic material of claim 1, wherein the hemostatic material comprises a plurality of chitosan fiber layers.

10. A process for preparing a hemostatic material, the process comprising:
a) providing a first chitosan fiber layer;
b) applying a solution of a weak acid to the first chitosan fiber layer;
c) depositing microporous polysaccharide microspheres on the first chitosan fiber layer;
d) depositing a therapeutic agent on the first chitosan fiber layer; and
e) placing a second chitosan fiber layer atop the first chitosan fiber layer upon which the microporous polysaccharide microspheres and the therapeutic agent are deposited, whereby a hemostatic material is obtained.

11. The process of claim 10, wherein steps a) through e) are repeated a plurality of times.

12. The process of claim 10, further comprising:
compressing the hemostatic material between a first surface and a second surface; and
heating the compressed hemostatic material, whereby a dry hemostatic material is obtained.

13. The process of claim 10, wherein the hemostatic material comprises from about 10 wt. % to about 50 wt. % microporous polysaccharide microspheres.

14. A method of controlling bleeding from a venous laceration, a venous puncture, an arterial laceration, or an arterial puncture, the method comprising:
applying a hemostatic material to the laceration or the puncture, whereby bleeding is controlled, the hemostatic material comprising a hemostatic agent and a therapeutic agent deposited on a hemostatic substrate, wherein the hemostatic substrate comprises chitosan.

15. The method of claim 14, wherein the hemostatic agent comprises microporous polysaccharide microspheres.

16. The method of claim 14, wherein the therapeutic agent is selected from the group consisting of an anti-inflammatory agent, an anti-infective agent, and an anesthetic.

17. The method of claim 14, wherein the chitosan comprises a fiber.

18. The method of claim 14, wherein the hemostatic material comprises from about 10 wt. % to about 50 wt. % of a hemostatic agent comprising microporous polysaccharide microspheres.

19. The method of claim 14, wherein the hemostatic material comprises a plurality of chitosan fiber layers.

20. A method of controlling oozing from a wound, the method comprising:
applying a hemostatic material to the oozing wound, the hemostatic material comprising a hemostatic agent and a therapeutic agent deposited on a hemostatic substrate, wherein the hemostatic substrate comprises chitosan, whereby oozing is controlled.

21. The method of claim 20, wherein the chitosan comprises a nonwoven fabric.

22. The method of claim 20, wherein the chitosan comprises a sponge.

23. The method of claim 20, wherein the hemostatic material comprises a plurality of chitosan fiber layers.

24. The method of claim 20, wherein the therapeutic agent is selected from the group consisting of an anti-inflammatory agent, an anti-infective agent, and an anesthetic.

25. The method of claim 20, wherein the therapeutic agent comprises a chemotherapy agent.

26. The method of claim 20, wherein the wound comprises a tumor bed.

27. The method of claim 20, wherein the wound comprises a liver wound.

28. The method of claim 20, wherein the wound comprises a brain wound.

29. A process for preparing a hemostatic material, the process comprising:
a) providing a first chitosan fiber layer;
b) applying a solution of a weak acid to the first chitosan fiber layer;
c) depositing microporous polysaccharide microspheres on the first chitosan fiber layer; and
d) placing a second chitosan fiber layer atop the first chitosan fiber layer upon which the microporous polysaccharide microspheres are deposited, whereby a hemostatic material is obtained.

30. The process of claim 29, wherein steps a) through d) are repeated a plurality of times.

31. The process of claim 29, further comprising:
heating the hemostatic material, whereby liquid is vaporized from the hemostatic material.

32. The process of claim 29, further comprising:
drying the hemostatic material.

33. The process of claim 29, further comprising:
compressing the hemostatic material between a first surface and a second surface; and
heating the compressed hemostatic material, whereby a dry hemostatic material is obtained.

34. The process of claim 33, wherein the first surface comprises a polytetrafluoroethylene and the second surface comprises a release paper.

35. The process of claim 29, wherein the hemostatic material comprises from about 10 wt. % to about 50 wt. % microporous polysaccharide microspheres.
